(19) United States
(12) Patent Application Publication
(10) Pub. No.: US 2010/0266952 A1
(43) Pub. Date: Oct. 21, 2010

(54) CYCLIC COMPOUND, PHOTORESIST BASE, PHOTORESIST COMPOSITION, MICROFABRICATION PROCESS, AND SEMICONDUCTOR DEVICE

(75) Inventors: Takashi Kashiwamura, Chiba (JP); Akinori Yomogita, Chiba (JP); Mitsuru Shibata, Chiba (JP); Takanori Owada, Chiba (JP); Hirotoshi Ishii, Chiba (JP); Masashi Sekikawa, Chiba (JP); Norio Tomotsu, Chiba (JP)

Correspondence Address: OBLON, SPIVAK, MCCLELLAND MAIER & NEUSTADT, L.L.P., 1940 DUKE STREET, ALEXANDRIA, VA, 22314, US

(73) Assignee: IDEMITSU KOSAN CO., LTD., TOKYO (JP)

(21) Appl. No.: 12/747,669

(22) PCT Filed: Dec. 11, 2008
(86) PCT No.: PCT/JP08/72468

§ 371 Date: Jun. 11, 2010

(30) Foreign Application Priority Data
Dec. 11, 2007 (JP) 2007-320224
Apr. 9, 2008 (JP) 2008-101108
Jun. 18, 2008 (JP) 2008-158769

(51) Int. Cl. G03F 7/004; C07C 69/76; C07C 59/64

(52) U.S. Cl. 430/270.1; 560/57; 562/468

(57) ABSTRACT

A cyclic compound shown by the following formula (I):

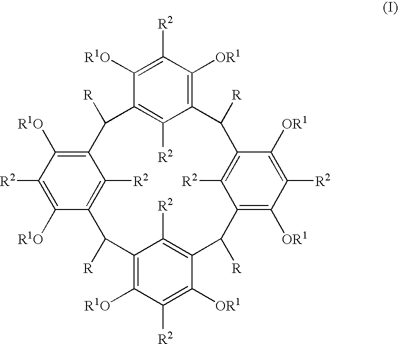

wherein, of two R¹s which are present on the same aromatic ring, one is a group shown by R³, and the other is a dissolution controlling group; R³s are independently hydrogen, a substituted or unsubstituted linear aliphatic hydrocarbon group having 1 to 20 carbon atoms, a substituted or unsubstituted branched aliphatic hydrocarbon group having 3 to 12 carbon atoms, a substituted or unsubstituted cyclic aliphatic hydrocarbon group having 3 to 20 carbon atoms, a substituted or unsubstituted aromatic group having 6 to 10 carbon atoms, an alkoxyalkyl group, a silyl group or a group formed by combining these groups with a divalent group.

### TECHNICAL FIELD

The invention relates to a novel cyclic compound, in particular, to a radiation-sensitive compound. The invention also relates to a photoresist base material used in the fields of electricity and electronics such as a semiconductor, the optical field or other fields, in particular to a photoresist base material for ultrafine processing.

### BACKGROUND ART

Lithography by extreme ultraviolet light (hereinafter often referred to as “EUVL”) or by an electron beam is useful as a fine processing method with a high productivity and a high resolution in the production of a semiconductor or the like. A photoresist having a high sensitivity and a high resolution for use in this lithography has been demanded. In respect of productivity, resolution or the like of a desired fine pattern, improvement in sensitivity of a photoresist is indispensable.

As for the photoresist used in the ultrafine processing using EUVL, a method is proposed in which a chemically amplified positive type photoresist which has a higher concentration of a photoacid generator than other resist compounds is used (for example, see Patent Document 1). However, as for a photoresist given as an example, in respect of line edge roughness, processing to a fineness of 100 nm, which is exemplified as a case where an electron beam is used, is thought to be the limit. The main reason therefor is assumed to be as follows. The three-dimensional morphology of a mass of polymer compounds or each molecule of polymer compounds, which is used as the base material, is large. Such large three-dimensional morphology exerts adverse effects on the production line width and the surface roughness.

The inventors already proposed a calixresorcinarene compound as a photoresist material which has a high sensitivity and a high resolution (see Patent Documents 2 and 3). Patent Document 4 also discloses a calixresorcinarene compound. However, part of these compounds appears to have insufficient solubility. In addition, this document does not describe the application of these compounds as a photoresist base material, and describes only the application of these compounds as an additive to be added to a photoresist base material which is composed of a known polymer. In the current semiconductor production processes, since a photoresist base material is dissolved in a solvent for film formation, a photoresist base material is required to be highly soluble in a solvent for coating. Therefore, the inventors also proposed a calixresorcinarene compound which has an improved solubility in a solvent for coating (see Patent Document 5).

Patent Document 1: JP-A-2002-055457

Patent Document 2: JP-A-2004-191913

Patent Document 3: JP-A-2005-075767

Patent Document 4: U.S. Pat. No. 6,093,517

Patent Document 5: JP-A-2007-197389

Although the solubility in a solvent for coating was increased and the processibility was improved by the above-mentioned technologies, improvement in resist pattern strength and adhesion with a substrate was demanded in order to conduct further fine processing of a resist pattern.

Further improvement in solubility in a solvent for coating has also been demanded.

An object of the invention is to provide a photoresist base material improved in solubility in a solvent for coating, resist pattern strength or adhesion with a substrate.

### DISCLOSURE OF THE INVENTION

According to the invention, the following cyclic compound, photoresist base material, or the like are provided.

1. A cyclic compound shown by the following formula (I):

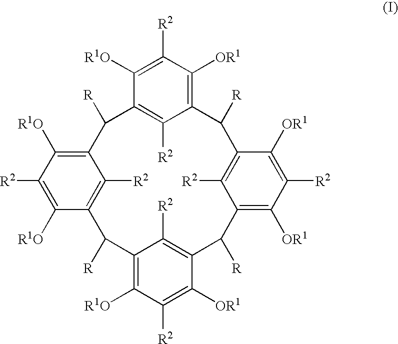

[wherein Rs are independently a group shown by the following formula (II):

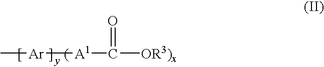

wherein Ar is an arylene group having 6 to 10 carbon atoms; a group formed by combining two or more arylene groups each having 6 to 10 carbon atoms, or a group formed by combining one or more arylene groups each having 6 to 10 carbon atoms with one or more selected from alkylene groups and ether groups;

A¹ is a single bond, an arylene group, an alkylene group, an ether group or a group formed by combining two or more selected from arylene groups, alkylene groups and ether bonds;

R³s are independently hydrogen, a substituted or unsubstituted linear aliphatic hydrocarbon group having 1 to 20 carbon atoms, a substituted or unsubstituted branched aliphatic hydrocarbon group having 3 to 12 carbon atoms, a substituted or unsubstituted cyclic aliphatic hydrocarbon group having 3 to 20 carbon atoms, a substituted or unsubstituted aromatic group having 6 to 10 carbon atoms, an alkoxyalkyl group, a silyl group, or a group formed by combining these groups with a divalent group, the divalent group being a substituted or unsubstituted alkylene group, a substituted or unsubstituted arylene group, a substituted or unsubstituted silylene group, a group formed by bonding of two or more of these groups, or a group formed by combining one or more of these groups with one or more selected from ester groups, carbonic ester groups and ether groups;

x is an integer of 1 to 5 and y is an integer of 0 to 3; and plural R³s, Ars, A¹s, xs and ys may be the same or different);

of two R¹s which are present on the same aromatic ring, one is a group shown by R³, and the other is a dissolution controlling group; and

R²s are independently hydrogen, a hydroxyl group, a group shown by OR³, a group shown by OR⁴ (wherein R⁴ is a dissolution controlling group), a linear aliphatic hydrocarbon group having 1 to 20 carbon atoms, a branched aliphatic hydrocarbon group having 3 to 12 carbon atoms, a cyclic aliphatic hydrocarbon group having 3 to 20 carbon atoms, an aromatic group having 6 to 10 carbon atoms or a group containing an oxygen atom].

2. The cyclic compound according to 1 wherein R² is hydrogen.
3. The cyclic compound according to 1 or 2, wherein OR³ is an acid-labile protecting group.
4. The cyclic compound according to 3, wherein the acid-labile protecting group is a substituent with a molecular weight of 15 or more and 2000 or less which has a tertiary aliphatic structure, an aromatic structure, a monocyclic aliphatic structure or a polycyclic aliphatic structure.
5. The cyclic compound according to 1 or 2, wherein OR³ is a group shown by one of the following formulas (III) to (VI):

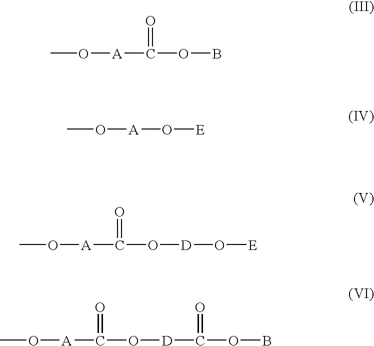

(wherein A is a substituted or unsubstituted linear aliphatic hydrocarbon group having 1 to 10 carbon atoms, a substituted or unsubstituted branched aliphatic hydrocarbon group having 3 to 10 carbon atoms, a substituted or unsubstituted cyclic aliphatic hydrocarbon group having 3 to 20 carbon atoms or a substituted or unsubstituted aromatic group having 6 to 10 carbon atoms;

B is a substituent having tertiary carbon as a bonding point with a tertiary aliphatic structure, an aromatic structure, a monocyclic aliphatic structure or a polycyclic aliphatic structure;

E is an aromatic structure, a monocyclic aliphatic structure, a polycyclic aliphatic structure or a substituent formed by combining at least one of an aromatic structure, a monocyclic aliphatic structure and a polycyclic aliphatic structure with a linear aliphatic hydrocarbon group having 1 to 10 carbon atoms; and

D is a substituted or unsubstituted linear aliphatic hydrocarbon group having 1 to 10 carbon atoms, a substituted or unsubstituted branched aliphatic hydrocarbon group having 3 to 10 carbon atoms, a substituted or unsubstituted cyclic aliphatic hydrocarbon group having 3 to 20 carbon atoms, or a substituted or unsubstituted aromatic group having 6 to 10 carbon atoms).

6. The cyclic compound according to 1 or 2, wherein OR³ is a group shown by one of the following formulas:

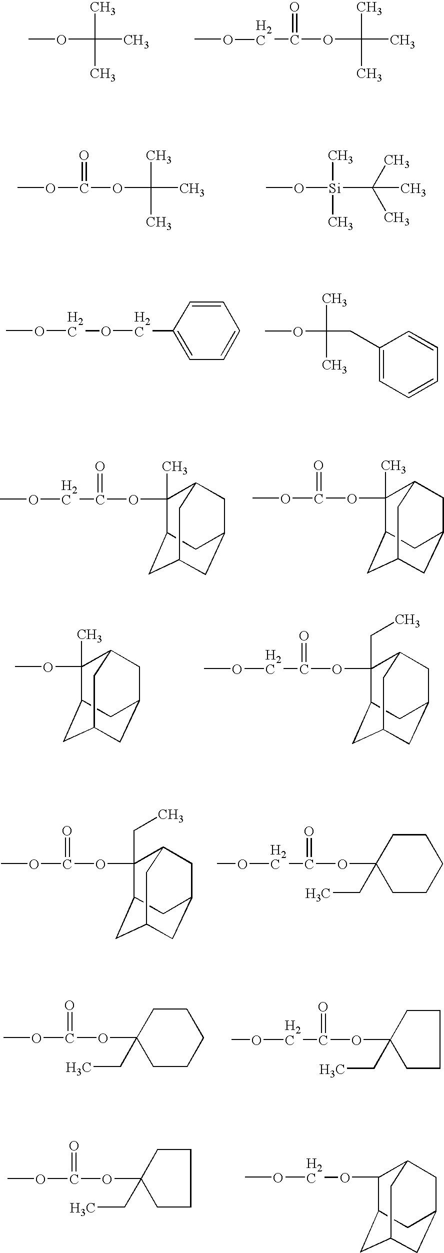

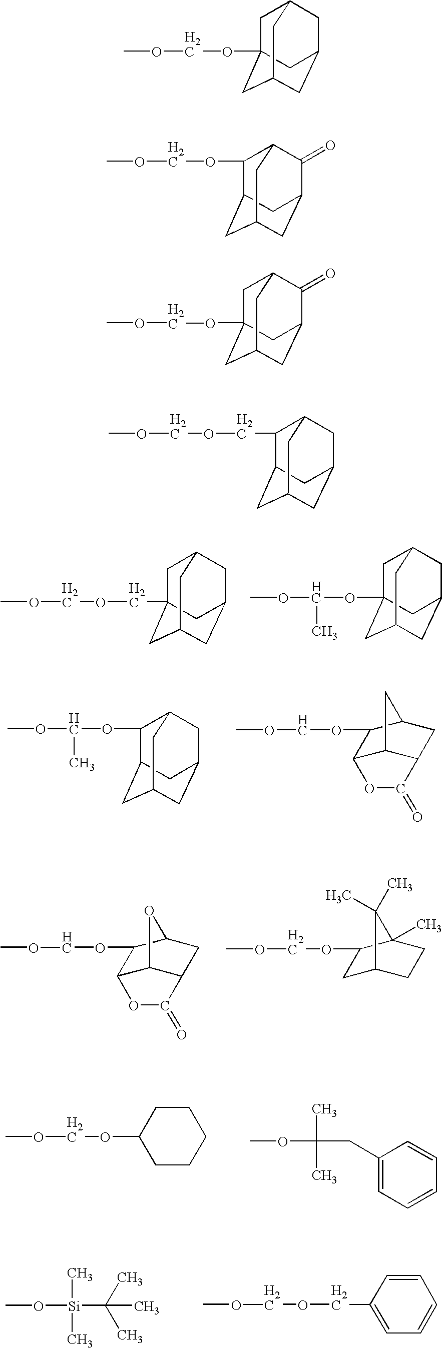

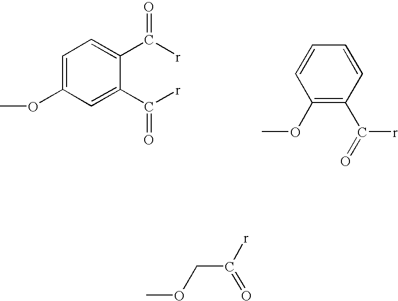

(wherein, rs are independently a substituent which does not have r of the substituents shown by the above formulas.)
7. The cyclic compound according to any one of 1 to 6, wherein the dissolution controlling group shown by R¹ and R⁴ is a substituted or unsubstituted linear aliphatic hydrocarbon group having 1 to 20 carbon atoms, a substituted or unsubstituted branched aliphatic hydrocarbon group having 3 to 12 carbon atoms, a substituted or unsubstituted cyclic aliphatic hydrocarbon group having 3 to 20 carbon atoms, a substituted or unsubstituted aromatic group having 6 to 10 carbon atoms, an alkoxyalkyl group, a silyl group, or a group formed by combining these groups with a divalent group, the divalent group being a substituted or unsubstituted alkylene group, a substituted or unsubstituted arylene group, a substituted or unsubstituted silylene group, a group formed by bonding two or more of these groups, or a group formed by bonding one or more of these groups with one or more groups selected from ester groups, carbonic ester groups and ether groups.
8. A photoresist base material comprising the cyclic compound according to any one of 1 to 7.
9. A photoresist composition comprising the photoresist base material according to 8 and a solvent.
10. The photoresist composition according to 9 further comprising a photoacid generator.
11. The photoresist composition according to 9 or 10 further comprising a basic organic compound as a quencher.
12. An ultrafine processing method using the photoresist composition according to any one of 9 to 11.
13. A semiconductor device prepared by the ultrafine processing method according to 12.
14. An apparatus comprising the semiconductor device according to 13.

The cyclic compound of the invention is easily dissolved in a solvent, since the dissolution controlling group is present on the aromatic ring.

Further, when the cyclic compound of the invention has a hydroxyl group on one aromatic ring, an intermolecular interaction by hydrogen bonding is increased. Therefore, when a fine pattern is produced by using the cyclic compound of the invention as a photoresist base, pattern strength and adhesion to a substrate is improved.

Further, when the cyclic compound of the invention has an acid-labile protecting group, since each molecule has a similar structure, a change in solubility of each molecule becomes uniform when desorption of the acid-labile protecting group occurs. As a result, end parts of a fine pattern become uniform, and as compared with convention patterns, the pattern has thinner convex parts. For this reason, a fine resist pattern can be formed.

### BRIEF DESCRIPTION OF THE DRAWINGS

FIG. 1 is a ¹H-NMR spectrum of the compound (1) synthesized in Example 1;

FIG. 2 is a ¹H-NMR spectrum of the compound (2) synthesized in Example 2;

FIG. 3 is a ¹H-NMR spectrum of the compound (3) synthesized in Example 3;

FIG. 4 is a ¹H-NMR spectrum of the intermediate (4′) synthesized in Example 4;

FIG. 5 is a ¹H-NMR spectrum of the compound (4) synthesized in Example 4;

FIG. 6 is a ¹H-NMR spectrum of the compound (5) synthesized in Example 5;

FIG. 7 is a ¹H-NMR spectrum of the compound (6) synthesized in Example 6; and

FIG. 8 is a ¹H-NMR spectrum of the compound (7) synthesized in Example 7.

### BEST MODE FOR CARRYING OUT THE INVENTION

The best mode for carrying out the invention will be explained hereinbelow.

The best mode for carrying out the invention is only one embodiment of the invention, and should not be construed as limiting the technical scope of the invention.

The cyclic compound of the invention has a structure shown by the following formula (I):

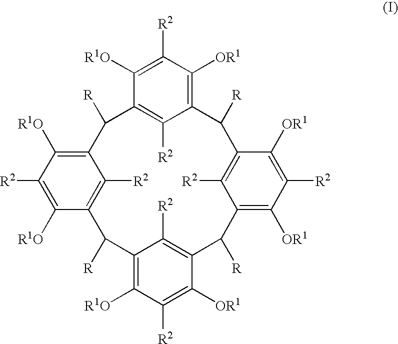

In the formula (I), Rs are independently a group shown by the following formula (II):

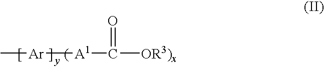

In the formula (II), Ar is an arylene group having 6 to 10 carbon atoms, a group formed by combining two or more arylene groups having 6 to 10 carbon atoms or a group formed by combining one or more arylene groups having 6 to 10 carbon atoms and one or more selected from alkylene groups and ether groups (—O—). Preferred examples include phenylene, methylphenylene, dimethylphenylene, trimethylphenylene, tetramethylphenylene, naphthylene, biphenylene and oxydiphenylene.

Of these, phenylene, biphenylene and oxydiphenylene are preferable.

A¹ is a single bond, an arylene group, an alkylene group, an ether group or a group formed by combining two or more selected from arylene groups, alkylene groups and ether groups. An alkylene group, an ether group or a group formed by combining one or more alkylene groups and one or more ether groups are preferable.

As the arylene group, the same groups as Ar can be given.

As the alkylene group, those having 1 to 4 carbon atoms such as a methylene group, a dimethylmethylene group, an ethylene group, a propylene group and a butylene group are preferable.

Preferred examples of the group formed by combining two or more of an alkylene group and an ether group include an oxymethylene group, an oxydimethylmethylene group, an oxyethylene group, an oxypropylene group and an oxybutylene group.

It is preferred that A¹ be a single bond or an oxymethylene group (—O—CH₂—).

R³s are independently hydrogen, a substituted or unsubstituted linear aliphatic hydrocarbon group having 1 to 20 carbon atoms, a substituted or unsubstituted branched aliphatic hydrocarbon group having 3 to 12 carbon atoms, a substituted or unsubstituted cyclic aliphatic hydrocarbon group having 3 to 20 carbon atoms, a substituted or unsubstituted aromatic group having 6 to 10 carbon atoms, an alkoxyalkyl group, a silyl group, or a group formed by bonding of these groups with a divalent group.

Preferred examples of the linear aliphatic hydrocarbon group having 1 to 20 carbon atoms include a methyl group, an ethyl group, a propyl group, a butyl group, a pentyl group, a hexyl group, a heptyl group and an octyl group.

Preferred examples of the branched aliphatic hydrocarbon group having 3 to 12 carbon atoms include a t-butyl group, an iso-propyl group, an iso-butyl group and a 2-ethylhexyl group.

Preferred examples of the cyclic aliphatic hydrocarbon group having 3 to 20 carbon atoms include a cyclohexyl group, a norbonyl group, an adamantyl group, a biadamantyl group and a diadamantyl group.

Preferred examples of the aromatic group having 6 to 10 carbon atoms include a phenyl group and a naphthyl group.

Preferred examples of the alkoxyalkyl group include a methoxymethyl group, an ethoxymethyl group and an adamantyloxymethyl group.

Preferred examples of the silyl group include a trimethylsilyl group and a t-butyldimethylsilyl group.

Each of the above groups may have a substituent. Specific examples thereof include an alkyl group such as a methyl group and an ethyl group, a ketone group, an ester group, an alkoxyl group, a nitrile group, a nitro group and a hydroxyl group.

R³ may be a group having a structure in which each of the above-mentioned groups is bonded with a divalent group.

Examples of the divalent group include a substituted or unsubstituted alkylene group, a substituted or unsubstituted arylene group, a substituted or unsubstituted silylene group, a group formed by bonding two or more of these groups or a group formed by combining one or more of these groups and one or more selected from ester groups (—CO₂—), carbonic ester groups (—CO₃—) and ether groups (—O—).

Preferred examples of the alkylene group include a methylene group and a methylmethylene group, and preferred examples of the arylene group include a phenylene group.

As the group formed by bonding two or more divalent group, a group having the following structure is preferable.

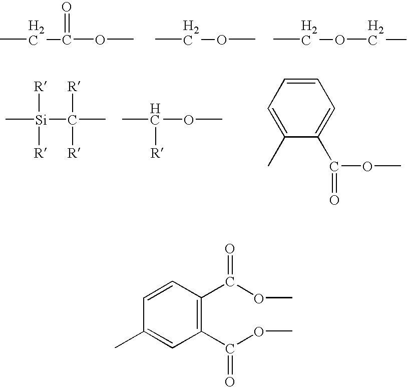

wherein R′s independently are H or an alkyl group.

OR³ is preferably an acid-labile protecting group, more preferably a substituent having a molecular weight of 15 or more and 2000 or less and having an aromatic structure, monocyclic aliphatic structure or polycyclic aliphatic structure.

Further, a cyclic compound of which OR³ is a group shown by one of the following formulas (III) to (VI) is preferable:

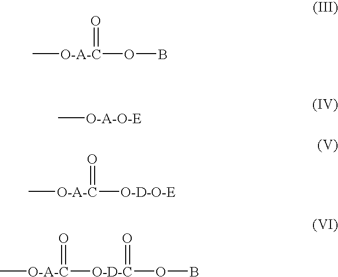

In the above formulas (III) to (VI), A is a substituted or unsubstituted linear aliphatic hydrocarbon group having 1 to 10 carbon atoms, a substituted or unsubstituted branched aliphatic hydrocarbon group having 3 to 10 carbon atoms, a substituted or unsubstituted cyclic aliphatic hydrocarbon group having 3 to 20 carbon atoms or a substituted or unsubstituted aromatic group having 6 to 10 carbon atoms;

B is a substituent with tertiary carbon as a bonding point having a tertiary aliphatic structure, an aromatic structure, a monocyclic aliphatic structure or a polycyclic aliphatic structure;

E is an aromatic structure, a monocyclic aliphatic structure and a polycyclic aliphatic structure or a group formed by combining at least one of an aromatic structure, a monocyclic aliphatic structure and a polycyclic structure with a linear aliphatic hydrocarbon group having 1 to 10 carbon atoms; and

D is a substituted or unsubstituted linear aliphatic hydrocarbon group having 1 to 10 carbon atoms, a substituted or unsubstituted branched aliphatic hydrocarbon group having 3 to 10 carbon atoms, a substituted or unsubstituted cyclic aliphatic hydrocarbon group having 3 to 20 carbon atoms or a substituted or unsubstituted aromatic group having 6 to 10 carbon atoms.

It is preferred that y in the formula (II) be 1. It is also preferred that x in the formula (II) be 1. Further, it is preferred that Ar be a phenyl group, and it is preferred that A^{1l} be a single bond.

Specific examples of the acid-labile protecting group (OR³) include groups shown by the following formulas:

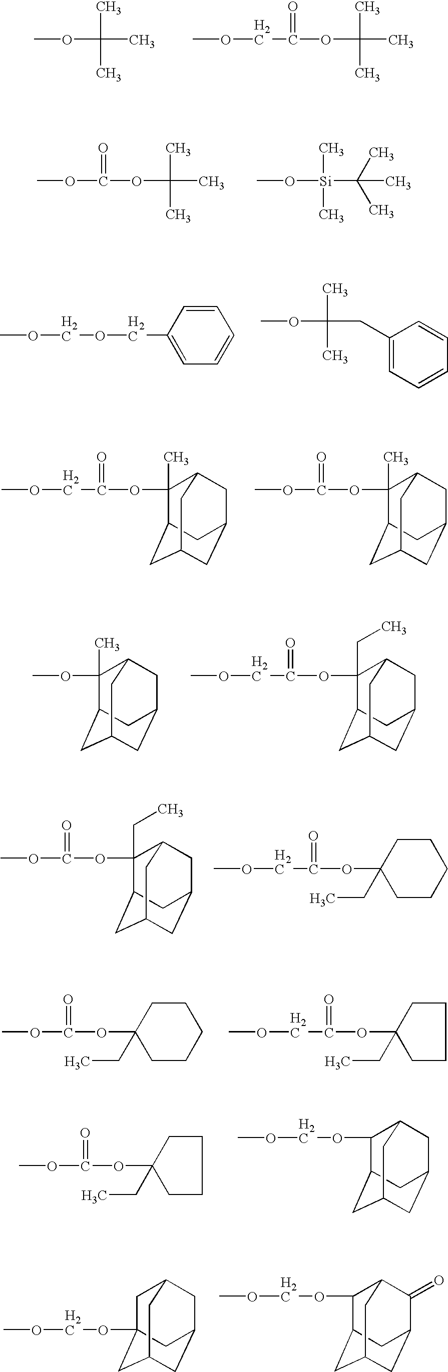

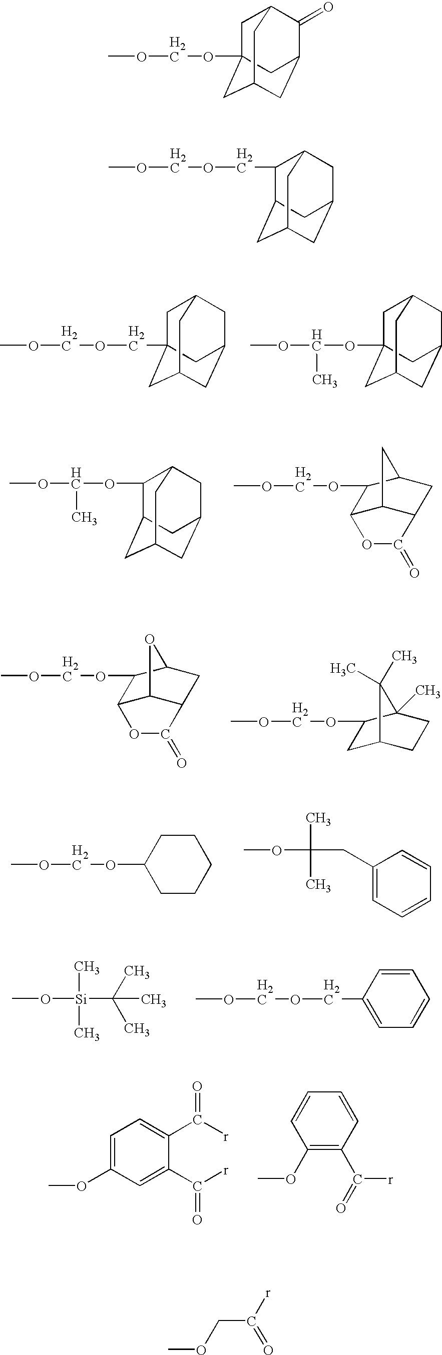

In the formulas, rs are independently a substituent which does not have r of the substituents shown by the above formulas.

x is an integer of 1 to 5, preferably an integer of 1 to 3.

y is an integer of 0 to 3, preferably 1 or 2.

Plural Rs are present in the formula (I). R³s, Ars, A¹s, xs and ys constituting R may be the same or different.

In the invention, it is preferred that the group shown by the formula (II) be one of the groups shown by the following formulas:

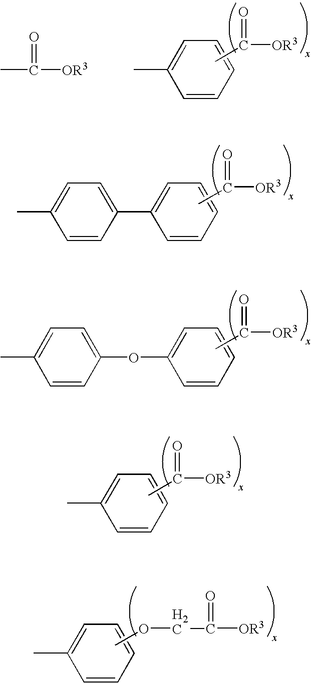

In the formula, R³ is the same group as in the formula (II), and x is an integer of 1 to 5.

In the formula (I), of two R¹s present on the same aromatic ring, one is a group shown by R³, and the other is a dissolution controlling group.

Of two R¹s, preferred examples of R¹ which is a group shown by R³ are the same as those of R³. A case where all of R³s present in R¹ are hydrogen and a case where R³s in R¹ are hydrogen and an acid-labile protecting group are particularly preferable. Specific examples of an acid-labile protecting group are the same as those mentioned above.

The dissolution controlling group is preferably a substituted or unsubstituted linear aliphatic hydrocarbon group having 1 to 20 carbon atoms, a substituted or unsubstituted branched aliphatic hydrocarbon group having 3 to 12 carbon atoms, a substituted or unsubstituted cyclic aliphatic hydrocarbon group having 3 to 20 carbon atoms, a substituted or unsubstituted aromatic group having 6 to 10 carbon atoms, an alkoxyalkyl group, a silyl group, or a group formed by combining these groups with a divalent group (a substituted or unsubstituted alkylene group, a substituted or unsubstituted arylene group, a substituted or unsubstituted silylene group, a group formed by bonding of two or more of these groups, or a group formed by bonding one or more of these groups with one or more groups selected from ester groups, carbonic ester groups and ether groups).

Preferred examples of each group of the dissolution controlling group are the same as those for R³ mentioned above.

R²s are independently hydrogen, a hydroxyl group, a group shown by OR³, a group shown by OR⁴ (R⁴ is a dissolution controlling group), a linear aliphatic hydrocarbon group having 1 to 20 carbon atoms, a branched hydrocarbon group having 3 to 12 carbon atoms, a cyclic aliphatic hydrocarbon group having 3 to 20 carbon atoms, an aromatic group having 6 to 10 carbon atoms or a group containing an oxygen atom.

Preferred examples of a linear aliphatic hydrocarbon group having 1 to 20 carbon atoms, a branched aliphatic hydrocarbon group having 3 to 12 carbon atoms, a cyclic aliphatic hydrocarbon group having 3 to 20 carbon atoms and an aromatic group having 6 to 10 carbon atoms are the same as those for R³ mentioned above. Preferred examples of the dissolution controlling group are the same as those for R¹ mentioned above.

As the group containing an oxygen atom, a group shown by OR³, a group shown by OR⁴ (R⁴ is a dissolution controlling group), an alkoxy group, an alkoxycarbonyl group or the like are preferable.

Preferably, R² is hydrogen.

Plural Rs, R¹s and R²s in the formula (I) may be the same or different.

The acid-labile protecting group has a high reactivity to EUVL and electron beams, and hence, it is improved in both sensitivity and etching resistance. Therefore, if the cyclic compound contains an acid-labile protecting group, the cyclic compound can be preferably used as a photoresist base material for ultrafine processing.

The cyclic compound of the invention can be synthesized by a known method. For example, in the presence of an acid catalyst, a fused cyclization reaction of an aldehyde compound having a corresponding structure and an aromatic compound having both a dissolution controlling group and a hydroxyl group is conducted to synthesize a calixresorcinarene derivative (precursor), and a compound corresponding to the groups such as R³ is introduced to the precursor by an esterification reaction, an etherification reaction, an acetalization reaction or the like. Specific synthesis examples will be explained in the following examples.

The cyclic compound according to the invention is useful as a photoresist base material, in particular as a photoresist base material used in ultrafine processing by lithography with extreme ultraviolet rays (wavelength: 15 nm or less), electron beams or the like.

When the cyclic compound according to the invention has one hydroxyl group and one dissolution controlling group at positions shown by OR¹, the cyclic compound has one hydroxyl group on one aromatic ring and four hydroxyl groups in one molecule in such a manner that they are three-dimensionally separated from each other. Accordingly, an intermolecular hydrogen bonding hardly occurs, and reinforcement by hydrogen bonding by an intermolecular interaction can be expected, whereby the strength of a fine pattern and adhesion with a substrate can be improved.

When the cyclic compound of the invention is used in a photoresist base material, it is preferable to remove basic impurities (for example, ammonia, alkaline metal ions such as Li, Na and K, an alkaline earth metal ions such as Ca and Ba) by purification. Specifically, the content of basic impurities is preferably 10 ppm or less, more preferably 2 ppm or less.

As the method for purification, washing with an aqueous acidic solution, an ion exchange treatment or re-precipitation using ultrapure water can be given. Purification may be conducted by combining these washing methods. For example, after washing with an aqueous acetic acid solution as the aqueous acetic solution, an ion exchange treatment or a re-precipitation treatment with ultrapure water is conducted.

Solubility of the cyclic compound of the invention in an alkaline developer is increased by the action of an acid. Therefore, it is preferred that the cyclic compound of the invention have an alkaline-soluble group.

Examples of the alkaline-soluble group include a hydroxyl group, a sulfonic acid group, a phenol group, a carboxyl group, a hexafluoroisopropanol group [—C(CF₃)₂OH] or the like. Preferred examples include a phenol group, a carboxy group and a hexafluoroisopropanol group, with a phenol group and a carboxyl group being further preferable.

acid-labile protecting group is a substituent which replaces a hydrogen atom of OH in the above-mentioned alkaline-soluble groups. Preferable examples thereof include —(CR₁₁ₐ)(R₁₂ₐ)(R₁₃ₐ), —C(R₁₄ₐ)(R₁₅ₐ)(OR₁₆ₐ) and —CO—OC(R₁₁ₐ)(R₁₂ₐ)(R₁₃ₐ).

Here, R₁₁ₐ to R₁₃ₐ are independently a substituted or unsubstituted alkyl group, cycloalkyl group, alkenyl group, aralkyl group or aryl group. R₁₄ₐ and R₁₅ₐ are independently a hydrogen atom or a substituted or unsubstituted alkyl group.

R₁₆ₐ is a substituted or unsubstituted alkyl group, cycloalkyl group, alkenyl group, aralkyl group or aryl group. Two of R₁₁ₐ, R₁₂ₐ and R₁₃ₐ or two of R₁₄ₐ, R₁₅ₐ and R₁₆ₐ may be bonded to form a ring.

The alkyl group, the cycloalkyl group and the aralkyl group of R₁₁ₐ to R₁₆ₐ may contain as a substituent a cycloalkyl group, a hydroxyl group, an alkoxy group, an oxo group, an alkylcarbonyl group, an alkyloxycarbonyl group, an alkylcarbonyloxy group, an alkylaminocarbonyl group, an alkylcarbonylamino group, an alkylsulfonyl group, an alkylsulfonyloxy group, an alkylsulfononylamino group, alkylaminosulfonyl group, an aminosulfonyl group, a halogen atom, a cyano group or the like.

The aryl group and the alkenyl group of R₁₁ₐ to R₁₃ₐ and R₁₆ₐ may contain as a substituent an alkyl group, a cycloalkyl group, a hydroxyl group, an alkoxy group, an oxo group, an alkylcarbonyl group, an alkyloxycarbonyl group, an alkylcarbonyloxy group, an alkylaminocarbonyl group, an alkylcarbonylamino group, an alkylsulfonyl group, an alkylsulfononyloxy group, an alkylsulfonylamino group, an alkylaminosulfonyl group, an aminosulfonyl group, a halogen atom, a cyano group or the like.

The alkyl group, the cycloalkyl group, the alkenyl group and aralkyl group of R₁₁ₐ to R₁₆ₐ each may have an ether group, a thioether group, a carbonyl group, an ester group, an amide group, an urethane group, an ureido group, a sulfonyl group or a sulfone group therein.

As the acid-labile protecting group, one having 4 or more carbon atoms in total is preferable. An acid-labile protecting group more preferably has 6 or more carbon atoms in total, further preferably 8 or more carbon atoms in total.

It is preferred that the acid-labile protecting group contain an alicyclic structure or an aromatic ring structure. As the alicyclic structure, a cyclopentane residue, a cyclohexane residue, a norbornane residue, an adamantane residue or the like can be given. As the aromatic ring structure, a benzene residue, a naphthalene residue, an anthracene residue or the like can be given.

These alicyclic structure and aromatic ring structure may have a substituent at an arbitral position.

Preferred specific examples of the acid-labile protecting group are given below. The invention is, however, not limited to these examples.

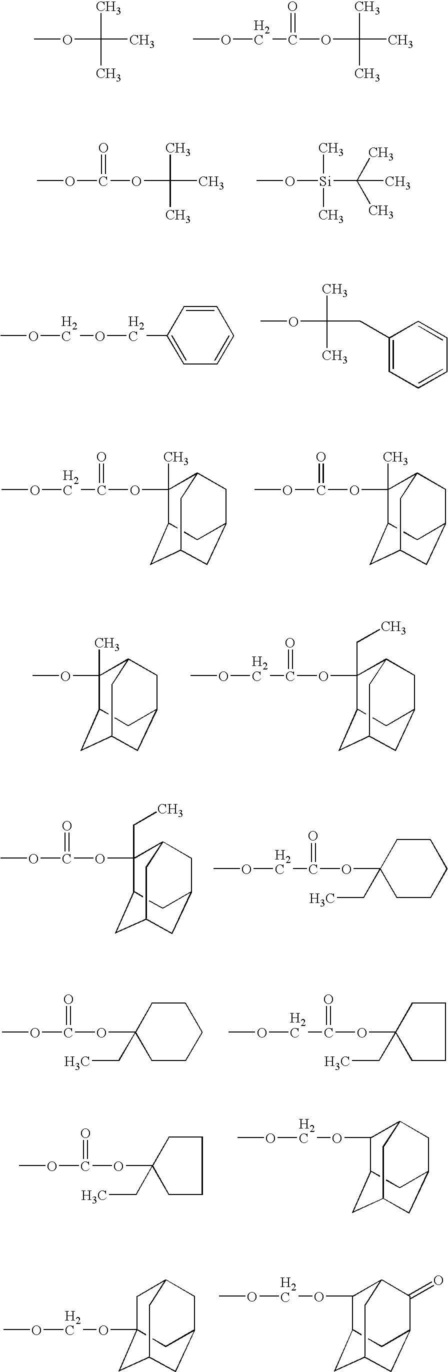

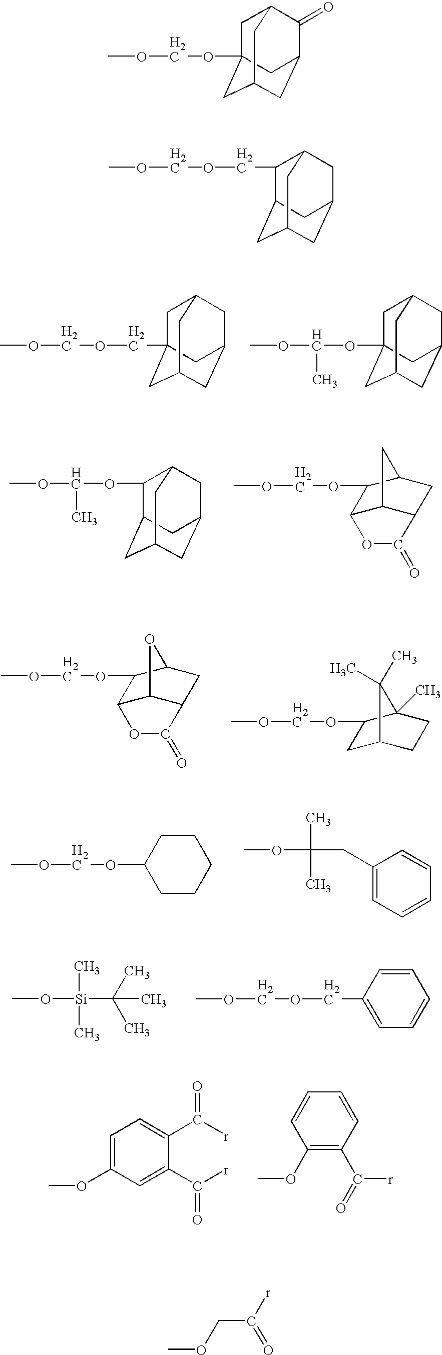

In the formulas, rs are independently a substituent which does not have r, of the substituents shown by the above formulas.

The above-mentioned cyclic compound can be used as a photoresist base material which is used in ultrafine processing by lithography with ultraviolet rays, electron beams or the like.

The photoresist composition of the invention contains the above-mentioned photoresist base material and a solvent.

The cyclic compound is contained in an amount of 50 to 99.9 wt %, more preferably 75 to 95 wt % of the total composition excluding the solvent. When the cyclic compound is used as the photoresist base material, it may be used singly or in combination of two or more as far as the advantageous effects of the invention are not impaired.

As the solvent to be used in the photoresist composition of the invention, for example, ethylene glycol monoalkyl ether acetates such as ethylene glycol monomethyl ether acetate and ethylene glycol monoethyl ether acetate; ethylene glycol monoalkyl ethers such as ethylene glycol monomethyl ether and ethylene glycol monoethyl ether; propylene glycol monoalkyl ether acetates such as propylene glycol monomethyl ether acetate (PGMEA) and propylene glycol monoethyl ether acetate; propylene glycol monoalkyl ethers such as propylene glycol monomethyl ether (PGME) and propylene glycol monoethyl ether; lactic acid esters such as methyl lactate and ethyl lactate (EL); aliphatic carboxylic acid esters such as methyl acetate, ethyl acetate, propyl acetate, butyl acetate and ethyl propionate (PE); other esters such as methyl 3-methoxypropionate, ethyl 3-methoxypropionate, methyl 3-ethoxypropionate and ethyl 3-ethoxypropionate; aromatic hydrocarbons such as toluene and xylene; ketones such as 2-heptane, 3-heptane, 4-heptane and cyclohexanone; cyclic ethers such as tetrahydrofuran and dioxane; and lactones such as γ-butyrolactone can be given. The solvent is not limited to those mentioned above. These solvents may be used singly or in combination of two or more.

The amount of other components other than the solvent in the composition, i.e. photoresist solid matters, is preferably an amount which is suitable for forming a photoresist layer in a desired thickness. Specifically, the photoresist solid matters are generally contained in an amount of 0.1 to 50 wt % of the total weight of the photoresist composition. However, it can be determined taking into consideration the kind of the base or solvent used, the desired thickness of the photoresist layer or the like. The solvent is contained preferably in an amount of 50 to 99.9 wt % of the total composition.

The photoresist composition of the invention may consist essentially of a photoresist base material of the cyclic compound of the invention and a solvent, or may consist of these components. The term “consist essentially of” means that the above-mentioned composition consists of a photoresist base material and a solvent but may contain other following additives in addition to these components.

The photoresist composition of the invention does not require an additive if the molecule of the base material contains a chromophore which is active to EUV and/or electron beams to allow the base material alone to exhibit activity as a photoresist. However, if there is a need to increase performance (sensitivity) as a photoresist, a photoacid generator (PAG) or the like is commonly contained as a chromophore.

There are no particular restrictions on the photoacid generator. Acid generators which have been proposed for a chemically amplification type resist can be used.

As such acid generators, many generators including onium salt-based acid generators such as iodonium salts and sulfonium salts, oxime sulfonate-based acid generators, diazomethane-based acid generators such as bisalkyl or bisarylsulfonyldiazomethanes and poly(bissulfonyl)diazomethanes, nitrobenzylsulfonate-based acid generators, iminosulfonate-based acid generators and disulfone-based acid generators.

As the onium salt-based acid generators, an acid generator shown by the following formula (b-0) can be used.

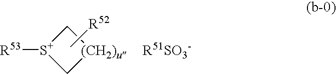

wherein R⁵¹ is a linear, branched or cyclic alkyl group or a linear, branched or cyclic fluorinated alkyl group; R⁵² is a hydrogen atom, a hydroxyl group, a halogen atom, a linear or branched alkyl group, a linear or branched halogenated alkyl group, or a linear or branched alkoxy group; R⁵³ is an aryl group which may have a substituent, and u″ is an integer of 1 to 3.

In the formula (b-0), R⁵¹ is a linear, branched or cyclic alkyl group or a linear, branched or a cyclic fluorinated alkyl group.

As the linear or branched alkyl group, one having 1 to 10 carbon atoms is preferable, one having 1 to 8 carbon atoms is further preferable, with one having 1 to 4 carbon atoms being most preferable.

As the cyclic alkyl group, one having 4 to 12 carbon atoms is preferable, one having 5 to 10 carbon atoms is further preferable, and one having 6 to 10 carbon atoms is most preferable.

As the fluorinated alkyl group, one having 1 to 10 carbon atoms is preferable, one having 1 to 8 carbon atoms is further preferable, and one having 1 to 4 carbon atoms is most preferable. The fluorination ratio of the fluorinated alkyl group (the ratio of the number of substituting fluorine atoms relative to the total number of hydrogen atoms in the alkyl group) is preferably 10 to 100% and further preferably 50 to 100%. In particular, a fluorinated alkyl group in which all of the hydrogen atoms are substituted by a fluorine atom is preferable since the strength of an acid is increased.

R⁵¹ is most preferably a linear alkyl group or a fluorinated alkyl group.

R⁵² is a hydrogen atom, a hydroxyl group, a halogen atom, a linear, branched or cyclic alkyl group, a linear or branched halogenated alkyl group or a linear or branched alkoxy group.

In R⁵², as the halogen atom, a fluorine atom, a bromine atom, a chlorine atom, an iodine atom or the like can be given, with a fluorine atom being preferable.

In R⁵², the alkyl group is a linear or branched alkyl group, of which the number of carbon atoms is preferably 1 to 5, more preferably 1 to 4, and most preferably 1 to 3.

In R⁵², the halogenated alkyl group is a group in which part or all of hydrogen atoms in the alkyl group are substituted by a halogen atom. The alkyl group thereof is the same “alkyl group” in R⁵² as mentioned above. As the halogen atom which substitutes the alkyl group, the same as those exemplified in the “halogen atom” mentioned above can be given. In the halogenated alkyl group, it is desired that 50 to 100% of the total number of hydrogen atoms be substituted by a halogen atom. It is more preferred that all of the hydrogen atoms be substituted.

In R⁵², the alkoxy group is a linear or branched alkoxy group, of which the carbon atoms is preferably 1 to 5, more preferably 1 to 4, and most preferably 1 to 3.

Of these, R⁵² is preferably a hydrogen atom.

R⁵³ is an aryl group which may have a substituent. As the basic ring structure excluding the substituent (host ring), a naphthyl group, a phenyl group, an anthracenyl group or the like can be given. In respect of the advantageous effects of the invention and absorption of irradiation light such as an ArF eximer laser light, a phenyl group is preferable.

As the substituent, a hydroxyl group, a lower alkyl group (a linear or branched lower alkyl group, the preferable number of carbon atoms of which is 5 or less, with a methyl group being preferable).

As the aryl group in R⁵³, one having no substituent is preferable.

u″ is an integer of 1 to 3, preferably 2 or 3, with 3 being particularly preferable.

As preferred examples of the acid generator shown by the formula (b-0), those shown by the following chemical formulas can be given.

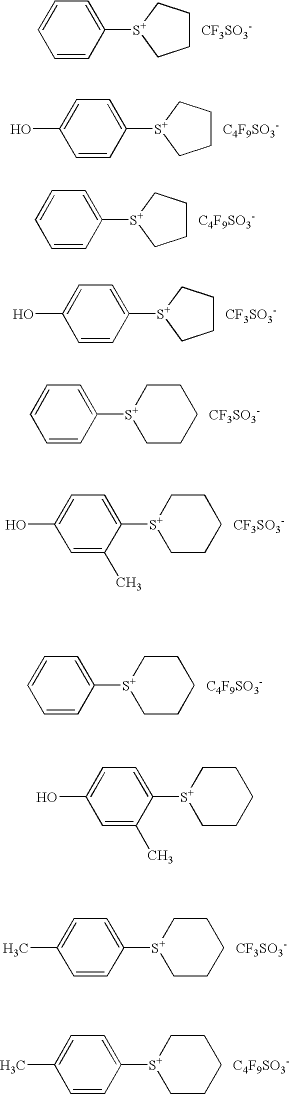

The acid generator shown by the formula (b-0) can be used singly or in a mixture of two or more.

As the other onium salt-based acid generator other than those shown by the formula (b-0), for example, compounds shown by the following formula (b-1) or (b-2) can be given.

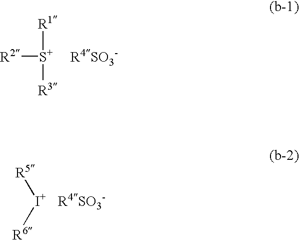

wherein R^{1″} to R^{3″}, R^{5″} and R^{6″} are independently a substituted or unsubstituted aryl group or alkyl group; R^{4″} is a linear, branched or cyclic alkyl group or fluorinated alkyl group; and at least one of R^{1″} to R^{3″} is an aryl group and at least one of R^{5″} and R^{6″} is an aryl group.

In formula (b-1), R^{1″} to R^{3″} are independently a substituted or unsubstituted aryl group or alkyl group. At least one of R^{1″} to R^{3″} is a substituted or unsubstituted aryl group. It is preferred that two or more of R^{1″} to R^{3″} be a substituted or unsubstituted aryl group. It is most preferred that all of R^{1″} to R^{3″} be a substituted or unsubstituted aryl group.

There are no particular restrictions on the aryl group shown by R^{1″} to R^{3″}. For example, an aryl group having 6 to 20 carbon atoms can be given. In the aryl group, part or all of hydrogen atoms may or may not be substituted by an alkyl group, an alkoxy group, a halogen atom or the like. As the aryl group, an aryl group having 6 to 10 carbon atoms is preferable since it can be synthesized at a low cost. As specific examples, a phenyl group, a naphthyl group or the like can be given.

As the alkyl group which is a substituent for the above-mentioned aryl group, an alkyl group having 1 to 5 carbon atoms is preferable, with a methyl group, an ethyl group, a propyl group, a n-butyl group and a tert-butyl group being most preferable.

As the alkoxy group which is a substituent for the aryl group, an alkoxy group having 1 to 5 carbon atoms is preferable, with a methoxy group and an ethoxy group being most preferable.

As the halogen atom which is a substituent for the aryl group, a fluorine atom is preferable.

There are no particular restrictions on the alkyl group shown by R^{1″} to R^{3″}. For example, a linear, branched or cyclic alkyl group having 1 to 10 carbon atoms or the like can be given. In respect of superior resolution, an alkyl group have 1 to 5 carbon atoms is preferable. Specific examples include a methyl group, an ethyl group, a n-propyl group, an isopropyl group, a n-butyl group, an isobutyl group, a n-pentyl group, a cyclopentyl group, a hexyl group, a cyclohexyl group, a nonyl group, a decanyl group or the like can be given. Of these, a methyl group is preferable due to improved resolution and synthesis at a low cost.

Of these, it is preferred that all of R^{1″} to R^{3″} be a phenyl group.

R^{4″} is a linear, branched or cyclic alkyl group or a fluorinated alkyl group.

As the linear or branched alkyl group, one having 1 to 10 carbon atoms is preferable, one having 1 to 8 carbon atoms is more preferable, and one having 1 to 4 carbon atoms is most preferable.

As the above-mentioned cyclic alkyl group, a cyclic group shown by the R¹ given above, of which the carbon atoms is preferably 4 to 15, more preferably 4 to 10 and most preferably 6 to 10, is preferable.

As the fluorinated alkyl group, one having 1 to 10 carbon atoms is preferable, one having 1 to 8 carbon atoms is further preferable, and one having 1 to 4 carbon atoms is most preferable. The fluorination ratio of the fluorinated alkyl group (the ratio of the fluorine atoms in the alkyl group) is preferably 10 to 100% and further preferably 50 to 100%. In particular, a fluorinated alkyl group in which all of the hydrogen atoms are substituted by a fluorine atom is preferable since the strength of an acid is increased.

R^{4″} is most preferably a linear or cyclic alkyl group or a fluorinated alkyl group.

In the formula (b-2), R^{5″} and R^{6″} are independently a substituted or unsubstituted aryl group or alkyl group. At least one of R^{5″} and R^{6″} is a substituted or unsubstituted aryl group. It is preferred that all of R^{5″} and R^{6″} be a substituted or unsubstituted aryl group.

As the substituted or unsubstituted aryl group shown by R^{5″} and R^{6″}, the same as those of the substituted or unsubstituted aryl group shown by R^{1″} to R^{3″} can be given.

As the alkyl group shown by R^{5″} and R^{6″}, the same as those of the alkyl group shown by R¹ to R^{3″} can be given.

Of these, it is most preferred that all of R^{5″} and R^{6″} be a phenyl group.

As for R^{4″} in the formula (b-2), the same as those for R^{4″} in the formula (b-1) can be given.

Specific examples of onium salt-based acid generators shown by the formulas (b-1) and (b-2) include trifluoromethanesulfonate or nonafluorobutanesulfonate of diphenyliodonium, trifluoromethanesulfonate or nonafluorobutanesulfonate of bis(4-tert-butylphenyl)iodonium, trifluoromethanesulfonate of triphenylsulfonium, heptafluoro propanesulfonate thereof or nonafluorobutanesulfonate thereof, trifluoromethanesulfonate of tri(4-methylphenyl)sulfonium, heptafluoropropanesulfonate thereof or nonafluorobutanesulfonate thereof, trifluoromethanesulfonate of dimethyl(4-hydroxynaphthyl)sulfonium, heptafluoropropaneslufonate thereof or nonafluorobutanesulfonate thereof, trifluoromethanesulfonate of monophenyldimethylsulfonium, heptafluoropropanesulfonate thereof or nonafluorobutanesulfonate thereof, trifluoromethanesulfonate of diphenylmonomethylsulfonium, heptafluoropropanesulfonate thereof or nonafluorobutanesulfonate thereof, trifluoromethanesulfonate of (4-methylphenyl)diphenylsulfonium, heptafluoropropanesulfonate thereof or nonafluorobutanesulfonate thereof, trifluoromethanesulfonate of (4-methoxyphenyl)diphenylsulfonium, heptafluoropropanesulfonate thereof or nonafluorobutanesulfonate thereof, trifluoromethanesulfonate of tri(4-tert-butyl)phenylsulfonium, heptafluoropropanesulfonate thereof or nonafluorobutanesulfonate thereof, trifluoromethanesulfonate of diphenyl(1-(4-methoxy)naphthyl)sulfonium, and heptafluoropropanesulfonate thereof or nonafluorobutanesulfonate thereof. Onium salts in which the anion portions thereof are replaced by methanesulfonate, n-propanesulfonate, n-butanesulfonate and n-octanesulfonate can be used.

In the above formula (b-1) or (b-2), an onium-based acid generator in which the anion portion thereof is replaced by an anion portion shown by the following formula (b-3) or (b-4) (the cation portion is similar to that in the formula (b-1) or (b-2)) can be used.

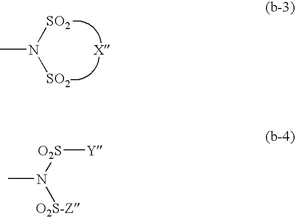

wherein X″ is an alkylene group having 2 to 6 carbon atoms in which at least one hydrogen atom is substituted by a fluorine atom, Y″ and Z″ are independently an alkyl group having 1 to 10 carbon atoms in which at least one hydrogen atom is substituted by a fluorine atom.

X″ is a linear or branched alkylene group in which at least one hydrogen atom is substituted by a fluorine atom, of which the number of carbon atoms is 2 to 6, preferably 3 to 5, and most preferably 3.

Y″ and Z″ are independently a linear or branched alkyl group in which at least one hydrogen atom is substituted by a fluorine atom, of which the number of carbon atoms is 1 to 10, preferably 1 to 7, and more preferably 1 to 3.

As for the number of carbon atoms in the alkylene group in X″ or the number of carbon atoms in the alkyl group in Y″ and Z″, a smaller number is preferable as far as it is within the above-mentioned range of the number of carbon atoms for the reason that the solubility in a resist solvent is good or the like.

In the alkylene group in X″ or the alkyl group in Y″ and Z″, a larger number of hydrogen atoms which are substituted by fluorine atoms is preferable since the acid strength is increased or the transparency to high-energy rays or electron beams with a wavelength of 200 nm or less is improved. The ratio of fluorine atoms in the alkylene group or the alkyl group, i.e. the fluorination ratio, is preferably 70 to 100% and further preferably 90 to 100%. A perfluoroalkylene group or a perfluoroalkyl group in which all of hydrogen atoms are substituted by fluorine atoms is most preferable.

In the invention, compounds shown by the following formulas (30) to (35) can also be used as the photoacid generator.

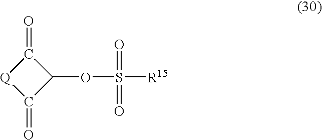

In the formula (30), Q is an alkylene group, an arylene group, an alkoxylene group and R¹⁵ is an alkyl group, an aryl group, a halogen-substituted alkyl group or a halogen-substituted aryl group.

It is preferred that the compound shown by the formula (30) be at least one selected from the group consisting of N-(trifluoromethylsulfonyloxy)succinimido, N-(trifluoromethylsulfonyloxy)phthalimide, N-(trifluoromethylsulfonyloxy)diphenylmaleimide, N-(trifluoromethylsulfonyloxy)bicyclo[2.2.1]hept-5-ene-2,3-dicarboximide, N-(trifluoromethylsulfonyloxy)naphthylimide, N-(10-camphorsulfonyloxy)succinimido, N-(10-camphorsulfonyloxy)phthalimide, N-(10-camphorsulfonyloxy)diphenylmaleimide, N-(10-camphorsulfonyloxy)bicyclo[2.2.1]hept-5-ene-2,3-dicarboximide, N-(10-camphorsulfonyloxy)naphthylimide, N-(n-octanesulfonyloxy)bicyclo[2.2.1]hept-5-ene-2,3-dicarboximide, N-(n-octanesulfonyloxy)naphthylimide, N-(p-toluenesulfonyloxy)bicyclo[2.2.1]hept-5-ene-2,3-dicarboximide, N-(p-toluenesulfonyloxy)naphthylimide, N-(2-trifluoromethylbenzenesulfonyloxy)bicyclo[2.2.1]hept-5-ene-2,3-dicarboximide, N-(2-trifluoromethylbenzenesulfonyloxy)naphthylimide, N-(4-trifluoromethylbenzenesulfonyloxy)bicyclo[2.2.1]hept-5-ene-2,3-dicarboximide, N-(4-trifluoromethylbenzenesulfonyloxy)naphthylimide, N-(perfluorobenzenesulfonyloxy)bicyclo[2.2.1]hept-5-ene-2,3-dicarboximide, N-(perfluorobenzenesulfonyloxy)naphtylimide, N-(1-naphthalenesulfonyloxy)bicyclo[2.2.1]hept-5-ene-2,3-dicarboximide, N-(1-naphthalenesulfonyloxy)naphthylimide, N-(nonafluoro-n-butanesulfonyloxy)bicyclo[2.2.1]hept-5-ene-2,3-dicarboximide, N-(nonafluoro-n-butanesulfonyloxy)naphthylimide, N-(perfluoro-n-octanesulfonyloxy)bicyclo[2.2.1]hept-5-ene-2,3-dicarboximide and N-(perfluoro-n-octanesulfonyloxy)naphthylimide.

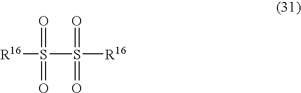

In the formula (31), R¹⁶s, which may be the same or different, are independently an optionally substituted linear, branched or cyclic alkyl group, an optionally substituted aryl group, an optionally substituted heteroaryl group or an optionally substituted aralkyl group.

It is preferred that the compound shown by the formula (31) be at least one selected from the group consisting of diphenyldisulfone, di(4-methylphenyl)disulfone, dinaphthyldisulfone, di(4-tert-butylphenyl)disulfone, di(4-hydroxyphenyl)disulfone, di(3-hydroxynaphthyl)disulfone, di(4-fluorophenyl)disulfone, di(2-fluorophenyl)disulfone and di(4-trifluoromethylphenyl)disulfone.

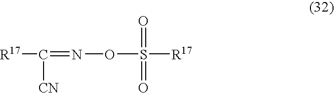

In the formula (32), R¹⁷s, which may be the same or different, are independently an optionally substituted linear, branched or cyclic alkyl group, an optionally substituted aryl group, an optionally substituted heteroaryl group or an optionally substituted aralkyl group.

It is preferred that the compound shown by the formula (32) be at least one selected from the group consisting of α-(methylsulfonyloxyimino)-phenylacetonitrile, α-(methylsulfonyloxyimino)-4-methoxyphenylacetonitrile, α-(trifluoromethylsulfonyloxyimino)-phenylacetonitrile, α-(trifluoromethylsulfonyloxyimino)-4-methoxyphenylacetonitrile, α-(ethysulfonyloxyimino)-4-methoxyphenylacetonitrile, α-(propylsulfonyloxyimino)-4-methylphenylacetonitrile and α-(methylsulfonyloxyimino)-4-bromophenylacetonitrile.

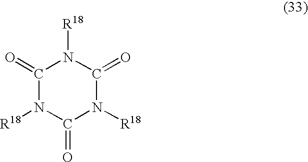

In the above-mentioned formula (33), R¹⁸s, which may be the same or different, are independently a halogenated alkyl group having one or more chlorine atom or one or more bromine atom. It is preferred that the halogenated alkyl group have 1 to 5 carbon atoms.

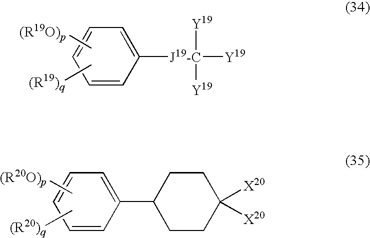

In the formulas (34) and (35), R¹⁹ and R²⁹ are independently an alkyl group having 1 to 3 carbon atoms such as a methyl group, an ethyl group, a n-propyl group and an isopropyl group, a cycloalkyl group such as a cyclopentyl group and a cyclohexyl group, an alkoxyl group having 1 to 3 carbon atoms such as a methoxy group, an ethoxy group and a propoxy group, or an aryl group such as a phenyl group, a toluoyl group and a naphthyl group, with an aryl group having 6 to 10 carbon atoms being preferable.

L¹⁹ and L²⁹ are independently an organic group having a 1,2-naphthoquinonediazide group. Specific preferable examples of the organic group having a 1,2-naphthoquinonediazide group include a 1,2-quinonediazidesulfonyl group such as a 1,2-naphthoquinonediazide-4-sulfonyl group, a 1,2-naphthoquinonediazide-5-sulfonyl group and 1,2-naphthoquinonediazide-6-sulfonyl group. In particular, a 1,2-naphthoquinonediazide-4-sulfonyl group and a 1,2-naphthoquinonediazide-5-sulfonyl group can be given.

p is an integer of 1 to 3, q is an integer of 0 to 4 and 1≦p+q≦5.

J¹⁹ is a single bond, a polymethylene group having 1 to 4 carbon atoms, a cycloalkylene group, a phenylene group, a group shown by the following formula (34a), or a group having a carbonyl bond, an ester bond, an amide bond or an ether bond.

Y¹⁹ is independently a hydrogen atom, an alkyl group or an aryl group, and X²⁰ is independently a group shown by the following formula (35a).

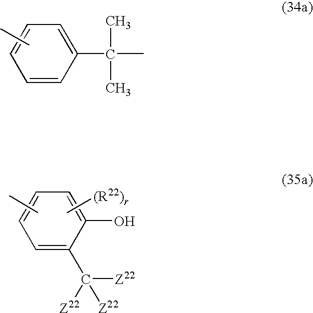

In the formula (35a), Z²² is independently an alkyl group, a cycloalkyl group or an aryl group and R²² is independently an alkyl group, a cycloalkyl group or an alkoxy group, and r is an integer of 0 to 3.

As other acid generators, bissulfonyldiazomethanes such as bis(p-toluenesulfonyl)diazomethane, bis(2,4-dimethylphenylsulfonyl)diazomethane, bis(tert-butylsulfonyl)diazomethane, bis(n-butylsulfonyl)diazomethane, bis(isobutylsulfonyl)diazomethane, bis(isopropylsulfonyl)diazomethane, bis(n-propylsulfonyl)diazomethane, bis(cyclohexylsulfonyl)diazomethane, bis(isopropylsulfonyl)diazomethane, 1,3-bis(cyclohexylsulfonylazomethylsulfonyl)propane, 1,4-bis(phenylsulfonylazomethylsulfonyl)butane, 1,6-bis(phenylsulfonylazomethylsulfonyl)hexane and 1,10-bis(cyclohexylsulfonylazomethylsulfonyl)decane, and halogen-containing triazine derivatives such as 2-(4-methoxyphenyl)-4,6-(bistrichloromethyl)-1,3,5-triazine, 2-(4-methoxynaphthyl)-4,6-(bistrichloromethyl)-1,3,5-triazine, tris(2,3-dibromopropyl)-1,3,5-triazine and tris(2,3-dibromopropyl)isocyanurate can be given.

Of these photoacid generators, a compound which generates an organic sulfonic acid by the action of activation rays or radiation rays is preferable.

The amount of a PAG is 0 to 40 wt %, preferably 5 to 30 wt % and further preferably 5 to 20 wt %, of the total composition excluding a solvent.

In the invention, an acid diffusion in a resist film controlling agent (quencher) which controls diffusion of an acid generated from the acid generator by irradiation of radiation rays to inhibit an unfavorable chemical reaction in an unexposed area may be contained in the photoresist composition. Due to the use of such an acid diffusion controlling agent, storage stability of the photoresist composition can be improved. Further, not only resolution is improved but also a change in line width caused by a change in waiting time before irradiation of electron beams or by a change in waiting time after irradiation of electron beams can be suppressed, whereby significant improvement is attained in processing stability.

Examples of the acid diffusion controlling agent include nitrogen atom-containing basic compounds such as monoalkylamine such as n-hexylamine, n-heptylamine, n-octylamine, n-nonylamine and n-decylamine; dialkylamine such as diethylamine, di-n-propylamine, di-n-heptylamine, di-n-octylamine and dicyclohexylamine; trialkylamine such as trimethylamine, triethylamine, tri-n-propylamine, tri-n-butylamine, tri-n-hexylamine, tri-n-pentylamine, tri-n-heptylamine, tri-n-octylamine, tri-n-nonylamine, tri-n-decanylamine and tri-n-dodecylamine; alkylalcholamine such as diethanolamine, triethanolamine, diisopropanolamine, triisopropanolamine, di-n-octanolamine and tri-n-octanolamine, and electron beam radiation decomposable basic compounds such as basic sulfonium compounds and basic iodonium compounds. These acid diffusion controlling agent may be used either singly or in combination of two or more.

The amount of the quencher is 0 to 40 wt %, preferably 0.01 to 15 wt %, of the total composition excluding a solvent.

In the invention, if necessary, miscible additives such as additive resins for improving the performance of a resist film, a surfactant for improving coating performance, a dissolution controlling agent, a sensitizer, a plasticizer, a stabilizer, a colorant, an anti-halation agent, a dye and a pigment can be appropriately added.

The dissolution controlling agent is a component which serves, when the solubility in an alkaline developer of the cyclic compound is too high, to lower the solubility to make the dissolution speed at the time of development appropriate.

As the dissolution controlling agent, for example, aromatic hydrocarbons such as naphthalene, phenanthrene, anthracene and acenaphthene; ketones such as acetophenone, benzophenone and phenylnaphthylketone; and sulfones such as methylphenylsulfone, diphenylsulfone and dinaphthylsulfone. Further, bisphenols into which an acid-labile functional group is introduced, tris(hydroxyphenyl)methane into which t-butylcarbonyl group is introduced or the like can be given. These dissolution controlling agents may be used singly or in combination of two or more. Although the amount of the dissolution controlling agent may be appropriately adjusted according to the type of the cyclic compound used, the amount is preferably 0 to 50 wt %, more preferably 0 to 40 wt %, and further preferably 0 to 30 wt % relative to the total weight of the solid matters.

The sensitizer is a component which serves to absorb energy of irradiated radiation rays, and transmits the energy to the acid generator, whereby the generated amount of an acid is increased. That is, the sensitizer is a component which improves apparent sensitivity of a resist. Although there are no particular restrictions on the sensitizer, the examples thereof include benzophenons, biacetyls, pyrenes, phenothiazines and fluorenes. These sensitizers can be used singly or in combination of two or more. The amount of the sensitizer is preferably 0 to 50 wt %, more preferably 0 to 20 wt % and further preferably 0 to 10 wt % of the total weight of solid components.

A surfactant is a component which serves to improve coating performance of the photoresist composition of the invention, to suppress the occurrence of striations and to improve developing properties as a resist. As such a surfactant, any of anionic surfactants, cationic surfactants, nonionic surfactants or amphoteric surfactants can be used. Of these surfactants, nonionic surfactants are preferable. Nonionic surfactants have good affinity with a solvent used in a photoresist composition, and hence, are more effective than other surfactants. Examples of the nonionic surfactants include polyoxyethylene higher alkyl ethers and polyoxyethylene higher alkylphenyl ethers and higher aliphatic acid diesters of polyethylene glycol. In addition to the above, in brand names, a series of products such as Eftop (manufactured by Jemco Co., Ltd.), Megafac (Dainippon Ink and Chemicals), Flurad (Sumitomo 3M, Ltd.), Asahi Guard and Surfrone (both are manufactured by Asahi Glass Co., Ltd.), Pepol (manufactured by Toho Chemical Industry Co., Ltd.), KP (manufactured by Shin-Etsu Chemical Co., Ltd.) and Polyflow (manufactured by Kyoeisha Chemical Co., Ltd.) can be given. Usable surfactants are not particularly limited. The amount of the surfactant is preferably 0 to 2 wt %, more preferably 0 to 1 wt % and further preferably 0 to 0.1 wt % of the total weight of the solid matters.

By adding a dye or a pigment, a latent image in an exposed part can be visualized, whereby adverse effects caused by halation at the time of exposure can be suppressed. Further, by adding an adhesive aid, adhesion with a substrate can be improved.

In order to prevent deterioration in sensitivity when an acid diffusion controlling agent is added, as well as to improve resist pattern shapes, waiting stability or the like, as an optional component, an organic carboxylic acid or an oxalic acid of phosphor or a derivative thereof can be contained. These compounds may be used in combination with an acid diffusion controlling agent or may be used singly.

As examples of an organic carboxylic acid, for example, malonic acid, citric acid, malic acid, succinic acid, benzoic acid, salicylic acid or the like are preferable. As the oxo acid of phosphor or its derivative, phosphoric acid or its derivatives such as esters thereof, such as phosphoric acid, phosphoric acid di-n-butyl esters and phosphoric acid diphenyl esters, phosphonic acid or its derivatives such as esters thereof, such as phosphonic acid, phosphonic acid dimethyl esters, di-n-butyl phosphate, phenylphosphonic acid, phosphonic acid diphenyl esters and phosphonic acid dibenzyl esters, and phosphinic acid or its derivatives such as esters thereof, such as phosphinic acid and phenylphosphinic acid. Of these, phosphonic acid is preferable.

In order to form a resist pattern, at first, on a substrate such as silicon wafer, gallium-arsenide wafer and aluminum-coated wafer, the photoresist composition of the invention is applied by coating methods such as rotary coating flow coating and roll coating, whereby a resist film is formed.

If necessary, the substrate is coated with a surface treatment agent in advance. As the surface treatment agent, a silane coupling agent such as hexamethylene disilazane (a hydrolysis polymerizable silane coupling agent having a polymerizable group or the like), an anchor coating agent or an undercoating agent (polyvinyl acetal, acrylic resins, vinyl acetate-based resins, epoxy resins, urethane resins or the like) and a coating agent obtained by mixing these undercoating agents and inorganic fine particles can be given.

If necessary, in order to prevent amines floating in the air from entering, a protective film may be formed on the resist film. By forming a protective film, a problem that an acid generated in a resist film by radiation rays is reacted with a compound which reacts with an acid such as an amine floating in the air as impurities, whereby the acid is deactivated to deteriorate a resist image and lower the sensitivity can be prevented. As the material for a protective film, a water-soluble and acidic polymer is preferable. For example, polyacrylic acid, polyvinylsulfonic acid or the like can be given.

In order to obtain a highly precise fine pattern, or to suppress the amount of an outgas generated during exposure, it is preferable to conduct heating before irradiation of radiation rays (before exposure). Although the heating temperature varies depending on the mixing ratio or the like of the photoresist composition, it is preferably 20 to 250° C., more preferably 40 to 150° C.

Subsequently, the resist film is exposed to radiation rays such as KrF eximer laser beams, extream ultraviolet rays, electron beams, X rays or the like, whereby the resist film is patterned into a desired shape. The exposure conditions or the like can be appropriately selected according to the mixing ratio or the like of the photoresist composition. In the invention, in order to form a highly precise fine pattern stably, it is preferred that heating be conducted after irradiation of radiation rays (after exposure). The heating temperature after the exposure (PEB) varies depending on the mixing ratio or the like of the photoresist composition, but it is preferably 20 to 250° C., more preferably 40 to 150° C.

Subsequently, by developing the exposed resist film with an alkaline developer, a desired resist pattern can be formed. As the alkaline developer, for example, an alkaline aqueous solution obtained by dissolving one or more of alkaline compounds such as mono-, di- or trialkylamines, mono-, di- or trialkanolamines, heterocyclic amines, tetramethylammonium hydroxide (TMAH) and choline in a concentration of preferably 1 to 10 wt %, more preferably 1 to 5 wt %. As the alkaline developer, alcohols such as methanol, ethanol and isopropyl alcohol or the above-mentioned surfactant can be added in an appropriate amount. Of these, it is particularly preferred that isopropyl alcohol be added in an amount of 10 to 30 wt %. If a developer comprising such an alkaline aqueous solution is used, generally, the resist film is washed with water after the development.

When the cyclic compound containing an acid-labile protecting group is used as a photoresist base material, by exposing the resist film with radiation rays such as KrF eximer laser beams, extream ultraviolet rays, electron beams or X rays into a desired shape, the acid-labile protecting group is removed or the structure thereof is changed. As a result, the resist film can be dissolved in an alkaline developer. It is preferred that an unexposed part of the pattern be not dissolved in an alkaline developer.

The non-dissolving properties for an alkaline developer cannot be determined easily, since preferable non-dissolving properties vary depending on the development conditions such as the size of a pattern to be formed and the kind of an alkaline developer or the like. However, when an aqueous 2.38% tetramethyl ammonium hydroxide solution is used as an alkaline developer, as for the non-dissolving properties, which are shown by the dissolution speed of a thin film formed of a photoresist base material in a developer, less than 1 nanometer/second is preferable, with less than 0.5 nanometer/second being particularly preferable.

If need arises, after the above-mentioned development in an alkaline developer, a post-baking treatment may be conducted, or an organic or inorganic reflection preventing film may be provided between the substrate and the resist film.

By conducting etching after the formation of a resist pattern, a substrate having a wiring pattern can be obtained. Etching can be conducted by a known method such as dry etching using a plasma gas, wet etching using an alkaline solution, a cupric chloride solution, a ferric chloride solution or the like. After the formation of a resist pattern, a plating treatment such as copper plating, solder plating, nickel plating and gold plating can be conducted.

A residual resist pattern after the etching can be peeled off by an aqueous solution having an alkaline property stronger than an organic solvent or an alkaline developer. Examples of the organic solvent include PGMEA, PGME, EL, acetone and tetrahydrofuran. As the strong alkaline solution, for example, a 1 to 20 wt % aqueous sodium hydroxide solution and a 1 to 20 wt % aqueous potassium hydroxide solution can be given. As the peeling method, for example, a dipping method, a spray method or the like can be given. A wiring substrate in which a resist pattern is formed may be a multilayer wiring substrate and may have a small through hole.

After a resist pattern is formed by using the photoresist composition of the invention, a wiring pattern may be formed by the lift off method in which a metal is deposited by vapor vacuum deposition, and the resist pattern is then eluted with a solution.

Ultrafine processing by lithography with extreme ultraviolet rays or electron beams can be conducted using the photoresist composition of the invention. According to the ultrafine processing method of the invention, a semiconductor device such as an ULSI, a mass storage memory device and an ultrahigh speed logic device can be produced.

According to the ultrafine processing method of the invention, the performance of the ULSI, the mass storage memory device, the ultrahigh speed logic device or the like can be significantly improved. Further, by incorporating a semiconductor device prepared by using the photoresist composition of the invention as a component, the performance of a semiconductor-built in product such as information home electric appliances, computer appliances and memory device appliances such as a USB memory and display appliances can be drastically improved.

### EXAMPLES

Examples will be given below, which will not limit the technical scope of the invention.

### Example 1

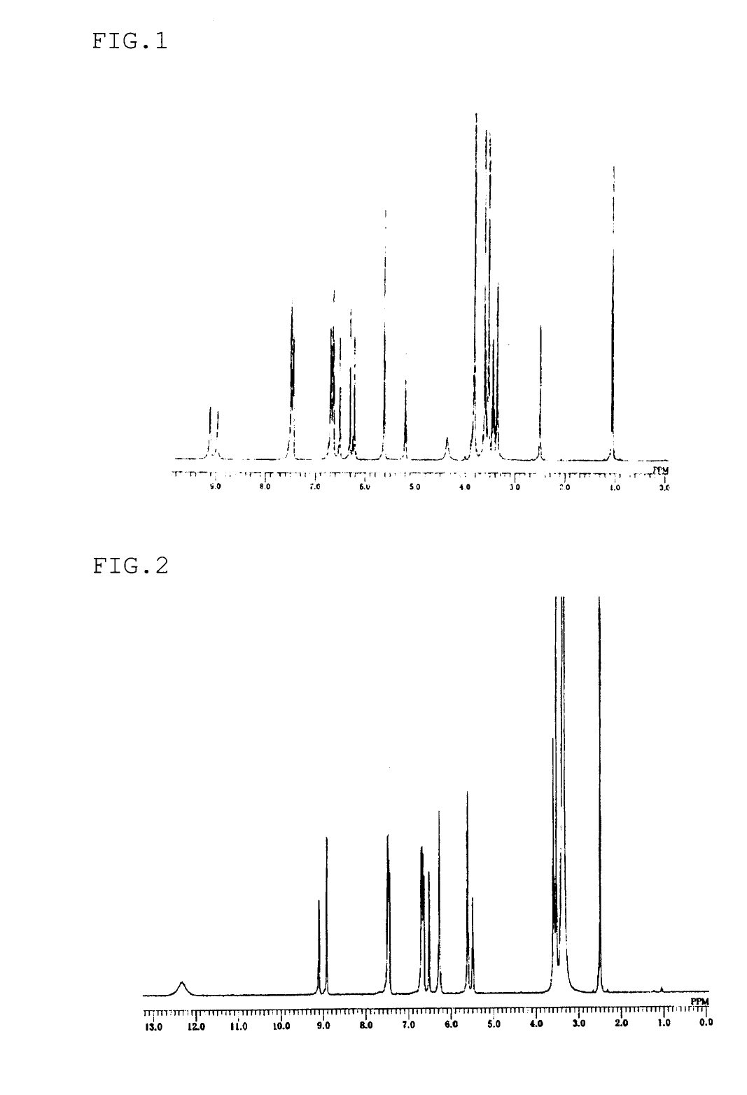

Under a nitrogen stream, in a round flask having a capacity of 200 mL, 10.0 g of 3-methoxyphenol (81 mmol), 13.2 g (80.6 mmol) of methyl 4-formylbenzoate and 100 mL of dehydrated dichloromethane were placed, and the mixture was cooled to −78° C. To this mixture, 30.8 mL (250 mmol) of boron trifluoride ether adduct was added dropwise. The mixture was heated to room temperature, and stirring was conducted continuously for 8 hours. The reaction solution was cooled to −78° C., and solids precipitated were washed with 80 mL of dichloromethane, 200 mL of water and ethanol, whereby the cyclic compound (1) was obtained in a yield of 20.5 g (yield: 94%). As a result of a ¹H-NMR measurement (FIG. 1), the cyclic compound was confirmed to have the following structure.

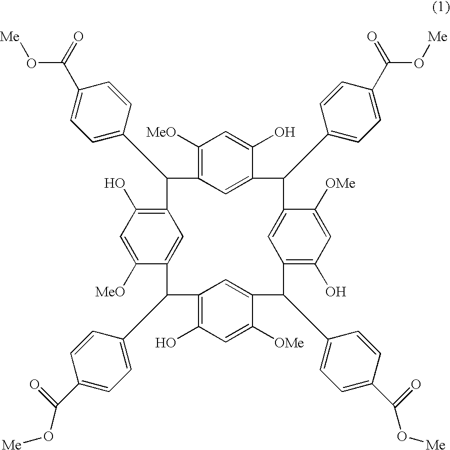

### Example 2

Under a nitrogen stream, to 0.8 g (0.74 mmol) of the cyclic compound (1) obtained in Example 1, 0.74 g (18.5 mmol) of sodium hydroxide and 10 mL of water were added. The resulting mixture was stirred while heating at 90° C. for 5 hours, and then allowed to cool. The reaction solution was made acidic by adding an aqueous solution of dilute hydrochloric acid. White precipitates deposited were filtered out and washed with water, whereby the cyclic compound (2) was obtained in a yield of 0.68 g (yield: 90%). As a result of a ¹H-NMR measurement (FIG. 2), the cyclic compound was confirmed to have the following structure.

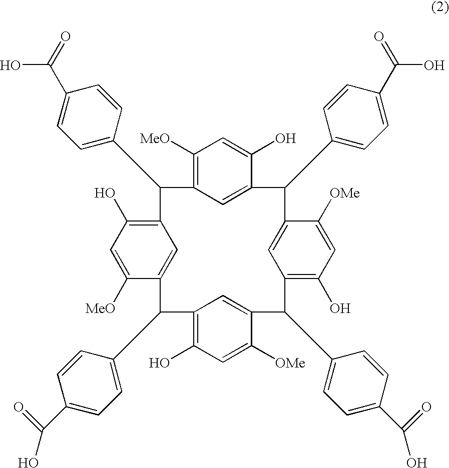

### Example 3

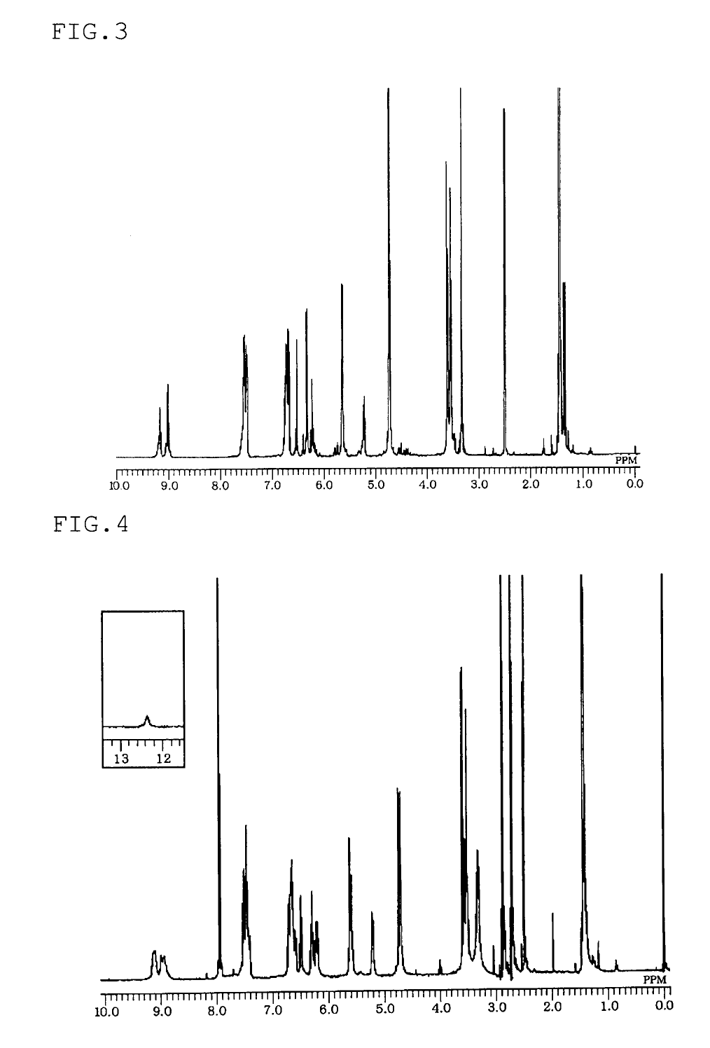

Under a nitrogen stream, to the mixture of 3.0 g (2.93 mmol) of the cyclic compound (2) obtained in Example 2, 1.64 g (15.5 mmol) of sodium carbonate and 100 mL of dimethylformamide, 5.04 g (15.5 mmol) of 2-tert-butyl bromoacetate was added dropwise. The resulting mixture was stirred while heating at 80° C. for 5 hours. The reaction mixture was allowed to cool, and water was added. White precipitates deposited were filtered out, whereby the cyclic compound (3) was obtained in a yield of 1.96 g (yield: 45%). As a result of a ¹H-NMR measurement (FIG. 3), the cyclic compound was confirmed to have the following structure.

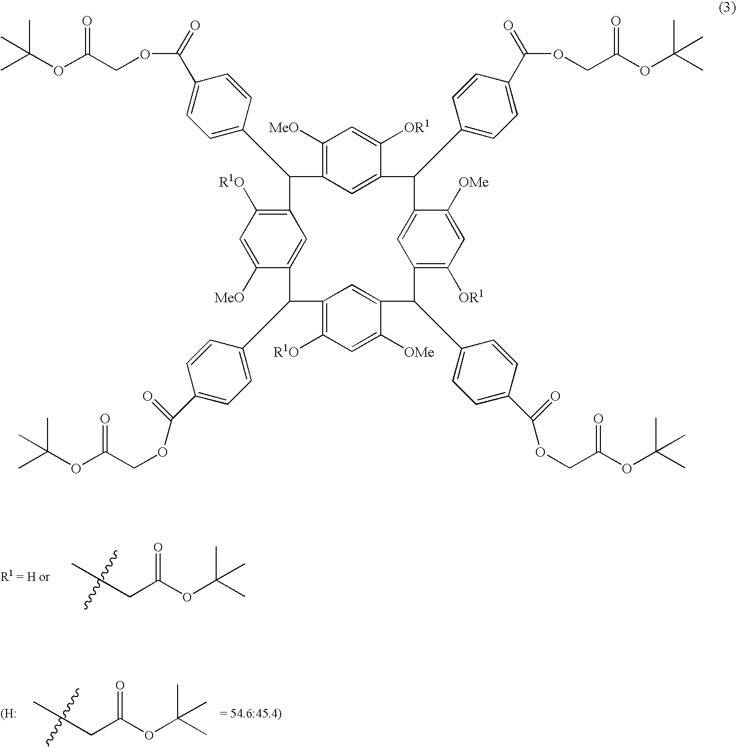

The resulting cyclic compound (3) was a mixture of a compound in which all of OR′s are a hydroxyl group, a compound in which one of four OR′s is substituted by an acid-labile protecting group and a compound in which two of four OR′s are substituted by an acid-labile protecting group. The amount ratio of the hydroxyl group and the acid-labile protecting group (hydroxyl group: acid-labile protecting group) in OR¹ in this mixture was 54.6:45.4.

### Example 4

Under a nitrogen stream, to the mixture of 10 g (9.8 mmol) of the cyclic compound (2) obtained in Example 2, 3.36 g (40 mmol) of sodium hydrogen carbonate and 120 mL of dimethylformamide, 7.8 g (40 mmol) of 2-tert-butyl bromoacetate was added dropwise. The resulting mixture was stirred while heating at 65° C. for 8 hours. The reaction mixture was allowed to cool, put into ion exchange water, and extracted with ethyl acetate. The ethyl acetate-extracted solution was concentrated and put in hexane. Solids deposited were filtered out, whereby a synthesis intermediate (4′) of the cyclic compound was obtained in an amount of 8.0 g (yield: 60%). As a result of a ¹H-NMR measurement (FIG. 4), the cyclic compound was confirmed to have the following structure.

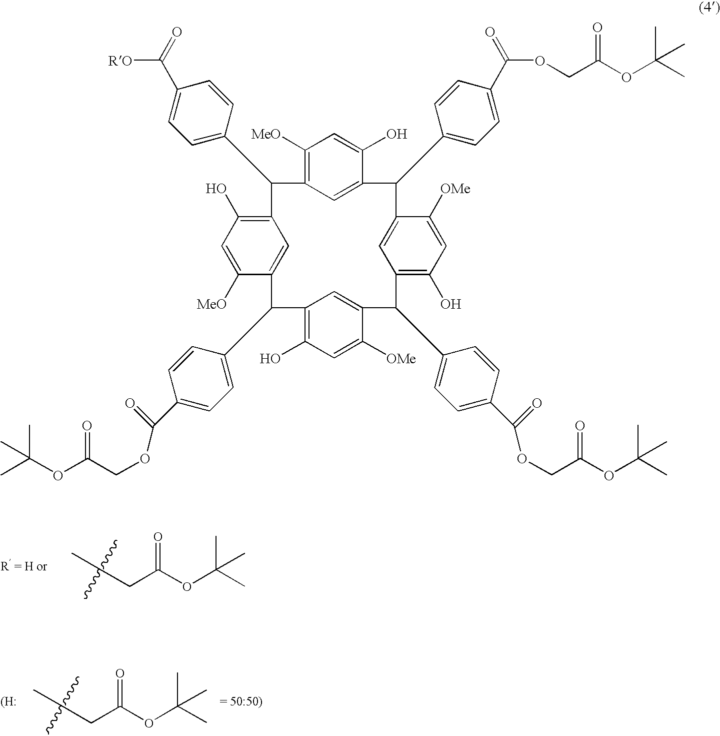

The resulting synthesis intermediate (4′) was a mixture of a synthesis intermediate in which OR was a hydroxyl group and a synthesis intermediate in which OR′ was an acid-labile protecting group. The mixing ratio of the synthesis intermediate in which OR′ was a hydroxyl group and the synthesis intermediate in which OR′ was an acid-labile protecting group was 50:50.

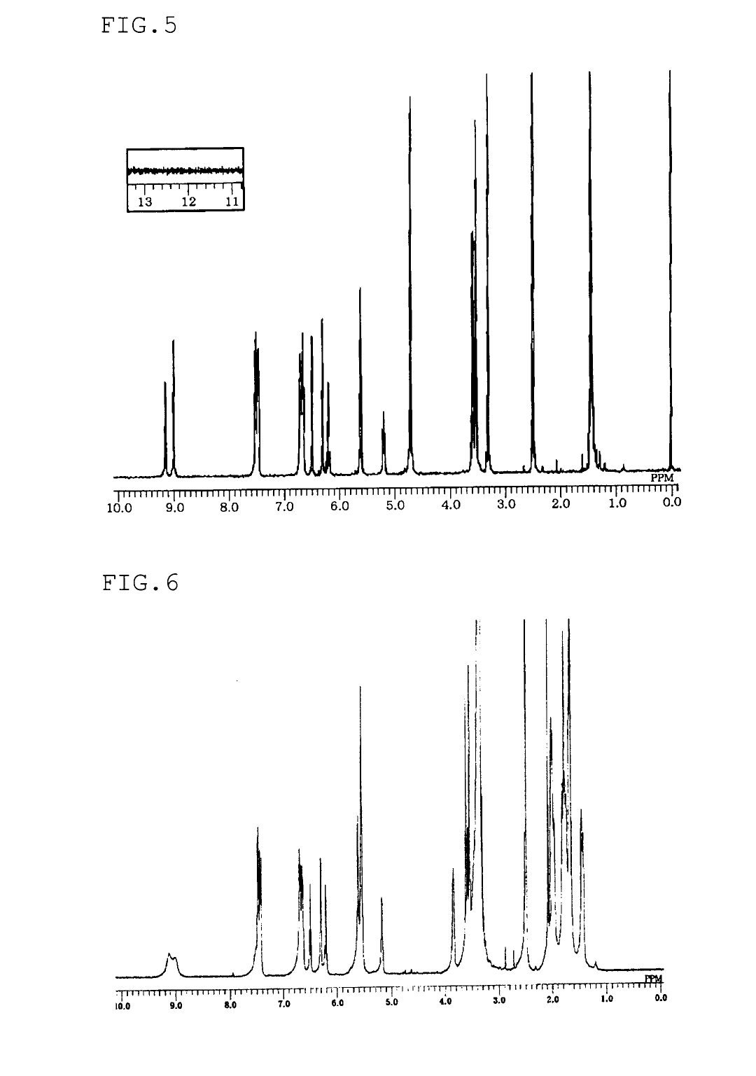

Under a nitrogen stream, to the mixture of 8.0 g (5.5 mmol) of the synthesis intermediate (4′) thus obtained, 0.20 g (0.34 mmol) of sodium hydrogen carbonate and 80 mL of dimethylformamide, 0.46 g (0.34 mmol) of 2-tert-butyl bromoacetate was added dropwise. The resulting mixture was stirred while heating at 65° C. for 4 hours. The reaction mixture was allowed to cool, put into ion exchange water, and extracted with ethyl acetate. The ethyl acetate-extracted solution was concentrated and re-precipitated from hexane. Solids deposited were filtered out, whereby a cyclic compound (4) was obtained in an amount of 5.0 g (yield: 58%). As a result of a ¹H-NMR measurement (FIG. 5), the cyclic compound was confirmed to have the following structure.

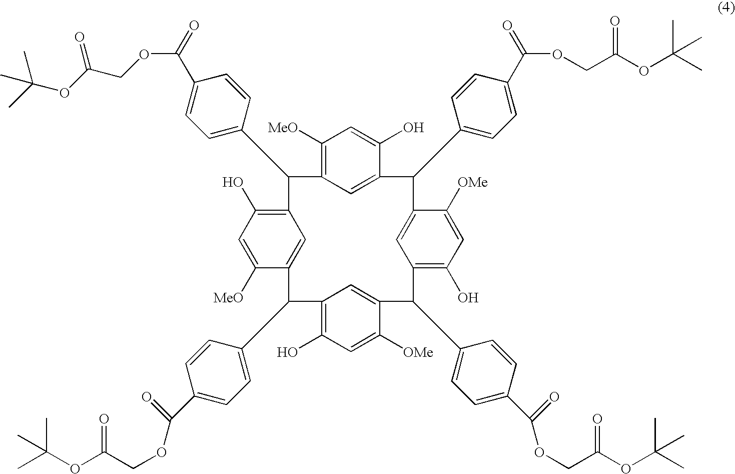

### Example 5

Under a nitrogen stream, to the mixture of 3.73 g (3.6 mmol) of the cyclic compound (2) obtained in Example 2, 3.65 g (36.1 mmol) of triethylamine and 100 mL of dimethylformamide, 3.62 g (18.1 mmol) of 2-chloromethoxyadamantane was added dropwise while cooling in ice bath. The resulting mixture was stirred at room temperature for 6 hours. Water was added to the reaction mixture, and solids deposited were filtered out, whereby a cyclic compound (5) was obtained in an amount of 4.33 g (yield: 71%). As a result of a ¹H-NMR measurement (FIG. 6), the cyclic compound was confirmed to have the following structure.

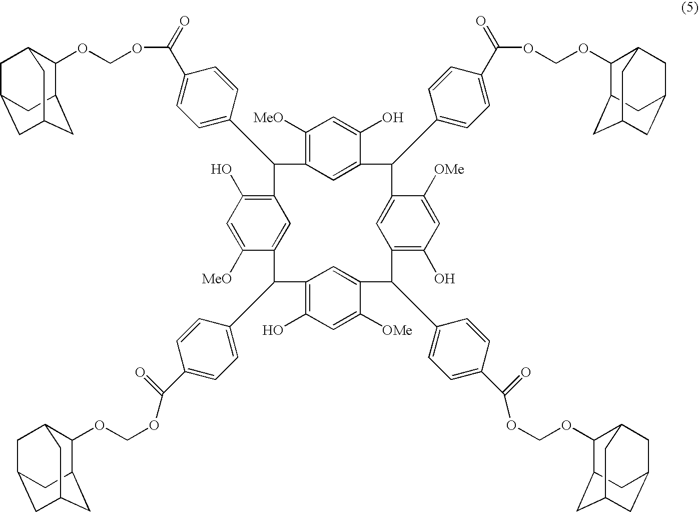

### Example 6

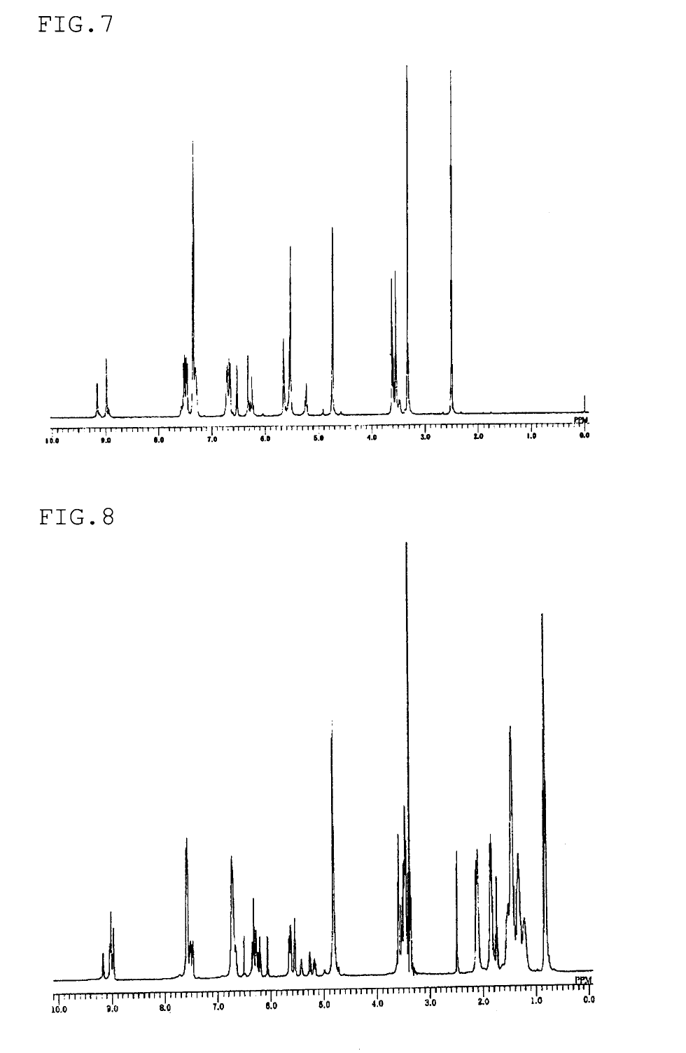

Under a nitrogen stream, to the mixture of 1.00 g (0.98 mmol) of the cyclic compound (2) obtained in Example 2, 0.99 g (9.75 mmol) of triethylamine and 30 mL of dimethylformamide, 0.77 g (4.9 mmol) of benzylchloromenthyl ether was added dropwise while cooling in ice bath. The resulting mixture was stirred at room temperature for 6 hours. Water was added to the reaction mixture, and solids deposited were filtered out, whereby a cyclic compound (6) was obtained in an amount of 0.63 g (yield: 43%). As a result of a ¹H-NMR measurement (FIG. 7), the cyclic compound was confirmed to have the following structure.

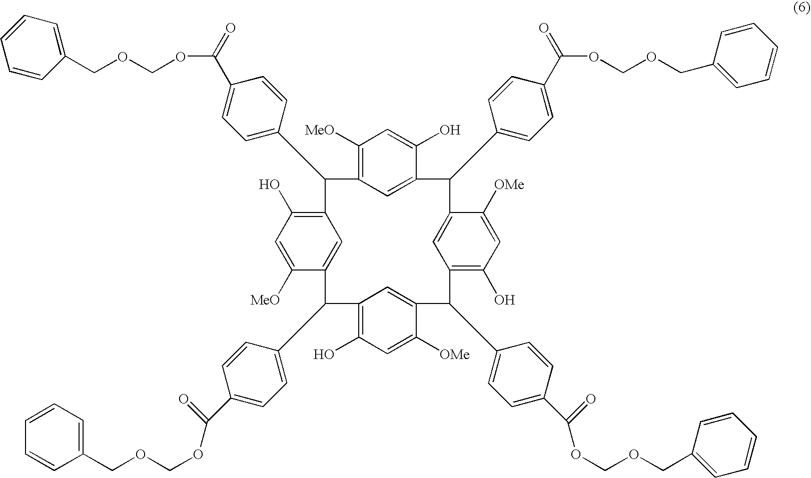

### Example 7

Under a nitrogen stream, to the mixture of 5.03 g (4.88 mmol) of the cyclic compound (2) obtained in Example 2, 1.86 g (21.95 mmol) of sodium hydrogen carbonate and 100 mL of N-methyl-2-pyrrolidone, 6.23 g (21.95 mmol) of 2-bromoacetate-1-ethylcyclohexyl was added. The resulting mixture was stirred while heating at 80° C. for 8 hours. The reaction mixture was allowed to cool, and extracted with ethyl acetate/water. An organic layer was concentrated and re-precipitated from hexane. Solids deposited were filtered out, whereby a cyclic compound (7) was obtained in an amount of 5.68 g (yield: 69%). As a result of a ¹H-NMR measurement (FIG. 8), the cyclic compound was confirmed to have the following structure.

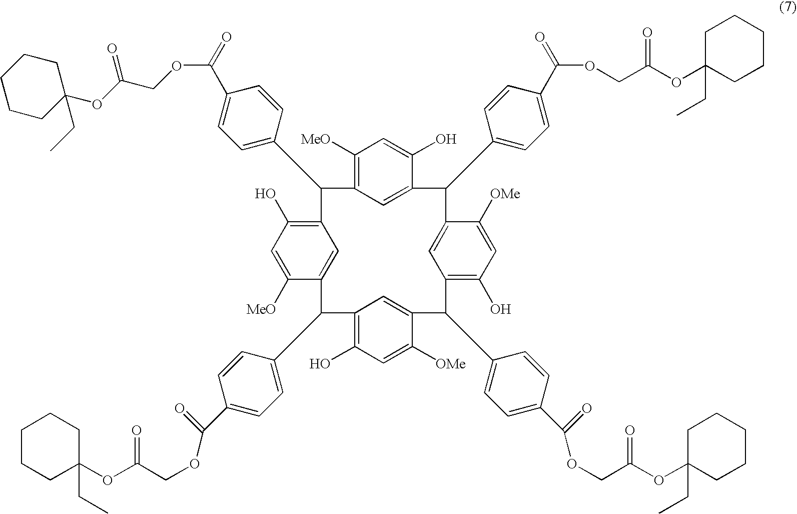

### Evaluation Example

A photoresist solution was prepared and a pattern was formed on silicon wafer using electron beams.

As a base material, 87 parts by weight of each of the cyclic compounds (3) to (7) synthesized in Examples 3 to 7, and, as a comparative example, 87 parts by weight of a cyclic compound shown by the following formula (8) was used. As a PAG, 10 parts by weight of triphenylsulfonium trifluoromethane sulfonate was used and 3 parts by weight of 1,4-diazabicyclo[2.2.2]octane was used as a quencher. By dissolving them in propylene glycol methylether acetate such that the concentration of these solid components became 5 wt %, whereby photoresist solutions using the cyclic compounds (3) to (8) as a base material were produced.

Each of these photoresist solutions was applied by spin coating on silicon water which had been subjected to a HMDS treatment, followed by heating at 100° C. for 180 seconds, whereby a thin film was formed. Subsequently, a substrate provided with this thin film was patterned by means of an electron beam lithography apparatus (accelerated voltage: 50 kV), followed by baking at 100° C. for 60 seconds. Then, development was conducted for 60 seconds in an aqueous tetrabutylammonium solution with a concentration of 2.38 wt %, followed by washing with pure water for 60 seconds. Thereafter, the substrate was dried in a nitrogen stream.

As a result, in each of the cases where a photoresist solution containing the cyclic compounds (3) to (7) as a base material was used, a 100 nm line-and-space pattern could be obtained in an excellent square shape with good linearity free from defects such as pattern collapse and pattern peeling.

Further, in the case where a photoresist solution containing the cyclic compound (3) as a base material was used, a 30 nm line-and-space pattern could be obtained in a sensitivity as high as 20 μc/cm², without significant defects such as pattern collapse and pattern peeling. In the case where the photoresist solution containing the cyclic compound (4) was used as a base material, a 35 nm line-and-space pattern could be obtained in a sensitivity as high as 20 μc/cm², without significant defects such as pattern collapse and pattern peeling. In the case where the photoresist solution using the cyclic compounds (5) to (7) as a base material were used, a 30 nm line-and-space pattern could be obtained in a sensitivity as high as 30 μc/cm², without significant defects such as pattern collapse and pattern peeling.

On the other hand, in the case where the photoresist solution containing as a base material the cyclic compound (8) was used as the comparative example, although a 100 nm-line-and-space pattern could be obtained in an excellent square shape with good linearity, pattern collapse and pattern peeling were observed.

A substrate having thereon the above-mentioned photoresist thin film was irradiated with EUV rays (wavelength: 13.5 nm) by means of an EUV exposure apparatus instead of an electron beam lithography apparatus. Thereafter, the substrate was baked at 100° C. for 90 seconds, and rinsed in a 2.38 wt % aqueous solution of hydrogenated tetramethylammonium for 30 seconds and in ion exchange water for 30 seconds, whereby a pattern was formed.

As a result of observation by means of a scanning electron microscope, as in the case where the electron beam lithography apparatus was used, in the case where the photoresist solution containing the cyclic compounds (3) to (7) as a base material were used, a 30 nm-line-and-space pattern could be obtained in an excellent square shape with good linearity, without defects such as pattern collapse and pattern peeling. On the other hand, in the case where the photoresist solution containing as a base material the cyclic compound (8) was used as the comparative example, as in the case where the electron beam lithography apparatus was used, although a 100 nm-line-and-space pattern could be obtained in an excellent square shape with good linearity, pattern collapse and pattern peeling were observed.

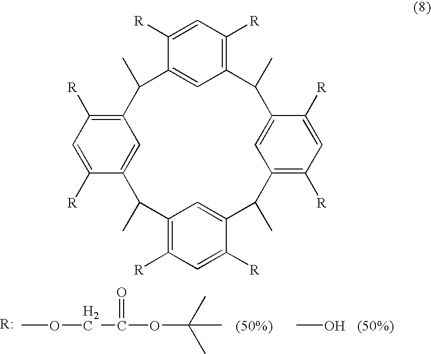

### INDUSTRIAL APPLICABILITY

The cyclic compound of the invention can be preferably used in a photoresist base material or a photoresist composition, in particular, in a photoresist base material or a photoresist composition for extreme ultraviolet rays and/or electron beams. Further, the cyclic compound of the invention can be used as an additive for adjusting solubility. The photoresist base material and the composition thereof are preferably used in electric/electronic fields such as semiconductor devices or optical fields.

The documents described in the specification are incorporated herein by reference in its entirety.

1. A cyclic compound represented by formula (I): 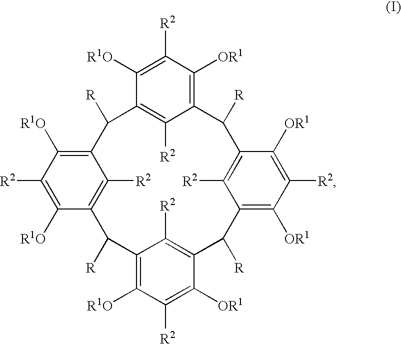
wherein, R, in each case, is independently a group represented by formula (II): 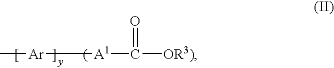
wherein, Ar is an arylene group comprising 6 to 10 carbon atoms, a group formed by combining two or more arylene groups each comprising 6 to 10 carbon atoms, or a group formed by combining one or more arylene groups each comprising 6 to 10 carbon atoms with at least one selected from the group consisting of an alkylene group and an ether group;
A¹ is a single bond, an arylene group, an alkylene group, an ether group or a group formed by combining at least two selected from the group consisting of an arylene group, an alkylene group, and an ether bond;
R³, in each case, is independently hydrogen, a substituted or unsubstituted linear aliphatic hydrocarbon group comprising 1 to 20 carbon atoms, a substituted or unsubstituted branched aliphatic hydrocarbon group comprising 3 to 12 carbon atoms, a substituted or unsubstituted cyclic aliphatic hydrocarbon group comprising 3 to 20 carbon atoms, a substituted or unsubstituted aromatic group comprising 6 to 10 carbon atoms, an alkoxyalkyl group, a silyl group, or a group formed by combining at least one substituted or unsubstituted linear aliphatic hydrocarbon group comprising 1 to 20 carbon atoms, substituted or unsubstituted branched aliphatic hydrocarbon group comprising 3 to 12 carbon atoms, substituted or unsubstituted cyclic aliphatic hydrocarbon group comprising 3 to 20 carbon atoms, substituted or unsubstituted aromatic group comprising 6 to 10 carbon atoms, alkoxyalkyl group, or silyl group
with
a divalent group, wherein the divalent group is a substituted or unsubstituted alkylene group, a substituted or unsubstituted arylene group, a substituted or unsubstituted silylene group, a group formed by bonding of two or more of a substituted or unsubstituted alkylene groups, a substituted or unsubstituted arylene groups, or a substituted or unsubstituted silylene groups, or a group formed by combining at least one substituted or unsubstituted alkylene group, substituted or unsubstituted arylene group, or substituted or unsubstituted silylene group with at least one selected from the group consisting of an ester group, a carbonic ester group, and an ether group;
x is an integer of 1 to 5;
y is an integer of 0 to 3;
R³, Ar, A¹, x, and y, in each case, may be the same or different;
of two R¹s present on a same aromatic ring, one is a group represented by R³, and the other is a dissolution controlling group; and
R² is in each case independently hydrogen, a hydroxyl group, a group represented by OR³, a group represented by OR⁴, wherein R⁴ is a dissolution controlling group, a linear aliphatic hydrocarbon group comprising 1 to 20 carbon atoms, a branched aliphatic hydrocarbon group comprising 3 to 12 carbon atoms, a cyclic aliphatic hydrocarbon group comprising 3 to 20 carbon atoms, an aromatic group comprising 6 to 10 carbon atoms or a group comprising an oxygen atom.

2. The cyclic compound according to claim 1 wherein R² is hydrogen.

3. The cyclic compound according to claim 1, wherein OR³ is an acid-labile protecting group.

4. The cyclic compound according to claim 3, wherein the acid-labile protecting group is a substituent with a molecular weight of 15 or more and 2000 or less, which has a tertiary aliphatic structure, an aromatic structure, a monocyclic aliphatic structure, or a polycyclic aliphatic structure.

5. The cyclic compound according to claim 1, wherein OR³ is a group represented by one of formulas (III) to (VI): 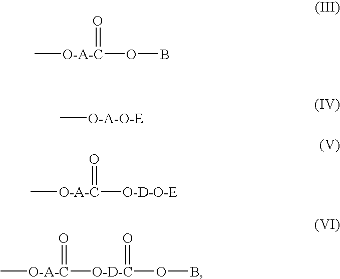
wherein:
A is a substituted or unsubstituted linear aliphatic hydrocarbon group comprising 1 to 10 carbon atoms, a substituted or unsubstituted branched aliphatic hydrocarbon group comprising 3 to 10 carbon atoms, a substituted or unsubstituted cyclic aliphatic hydrocarbon group comprising 3 to 20 carbon atoms or a substituted or unsubstituted aromatic group comprising 6 to 10 carbon atoms;
B is a substituent comprising tertiary carbon as a bonding point with a tertiary aliphatic structure, an aromatic structure, a monocyclic aliphatic structure, or a polycyclic aliphatic structure;
E is an aromatic structure, a monocyclic aliphatic structure, a polycyclic aliphatic structure, or a substituent formed by combining at least one of an aromatic structure, a monocyclic aliphatic structure, and a polycyclic aliphatic structure, with a linear aliphatic hydrocarbon group comprising 1 to 10 carbon atoms; and
D is a substituted or unsubstituted linear aliphatic hydrocarbon group comprising 1 to 10 carbon atoms, a substituted or unsubstituted branched aliphatic hydrocarbon group comprising 3 to 10 carbon atoms, a substituted or unsubstituted cyclic aliphatic hydrocarbon group comprising 3 to 20 carbon atoms, or a substituted or unsubstituted aromatic group comprising 6 to 10 carbon atoms.

6. The cyclic compound according to claim 1, wherein OR³ is 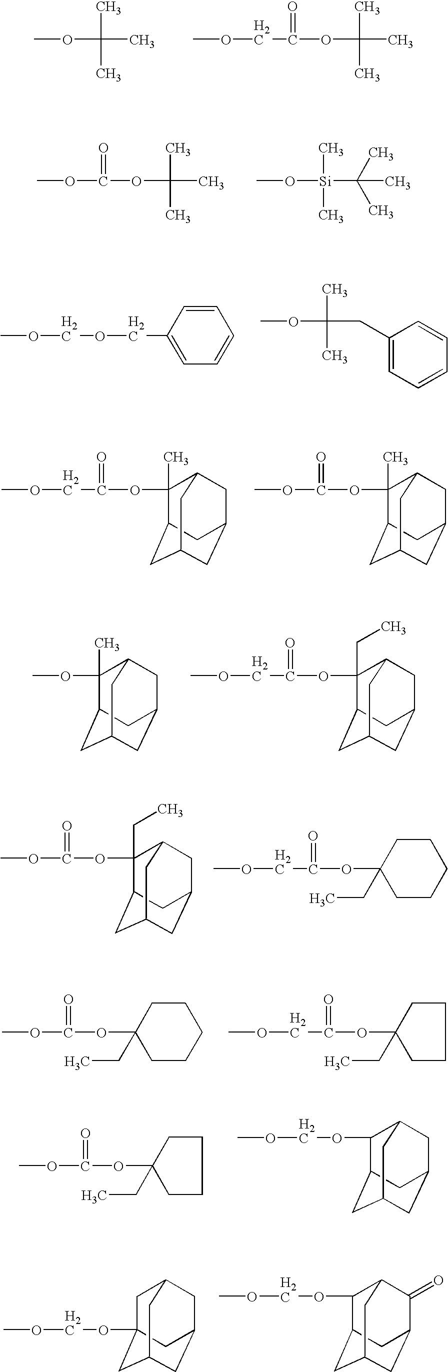 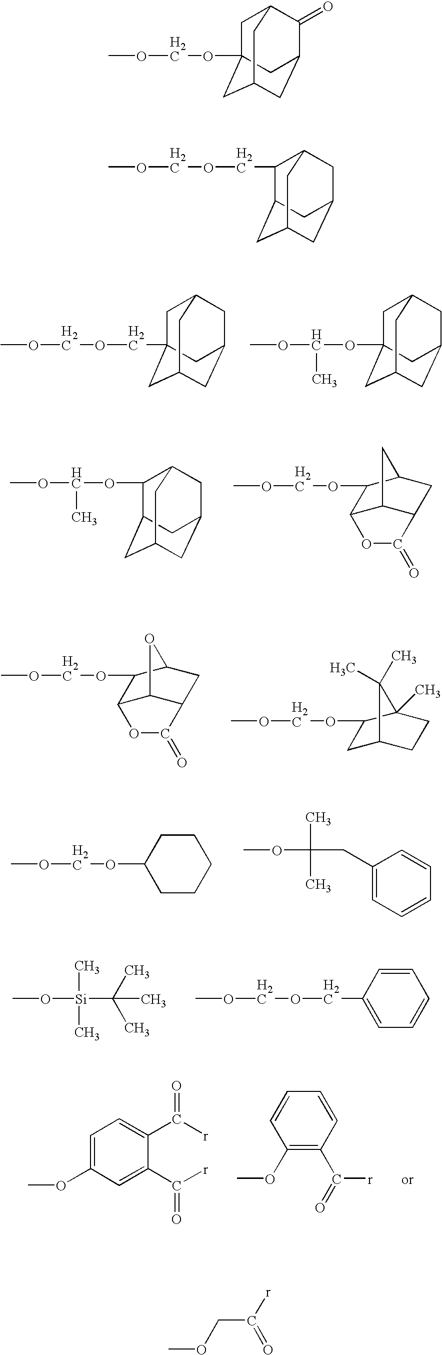
wherein, r is independently a substituent which does not comprise

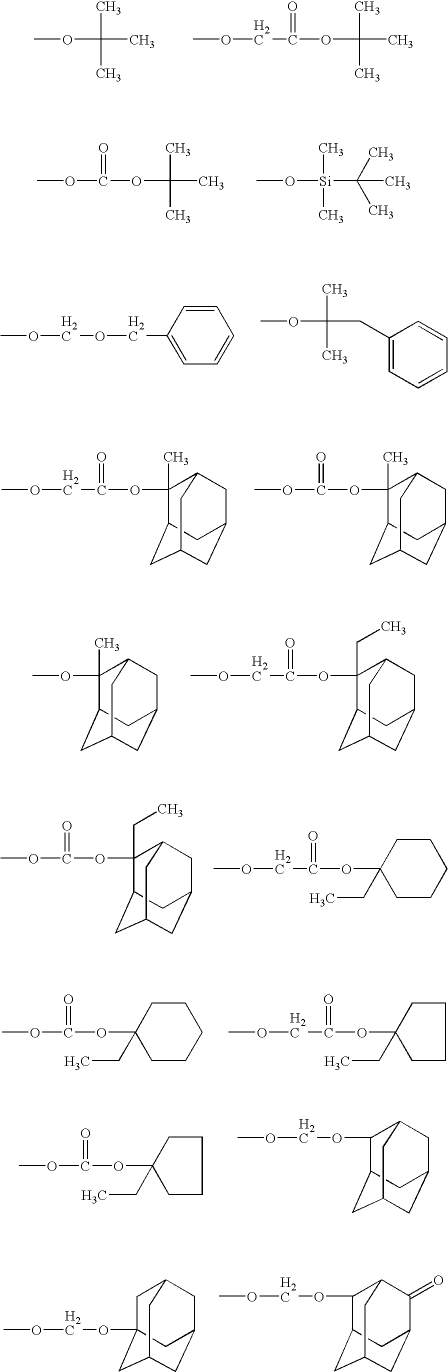

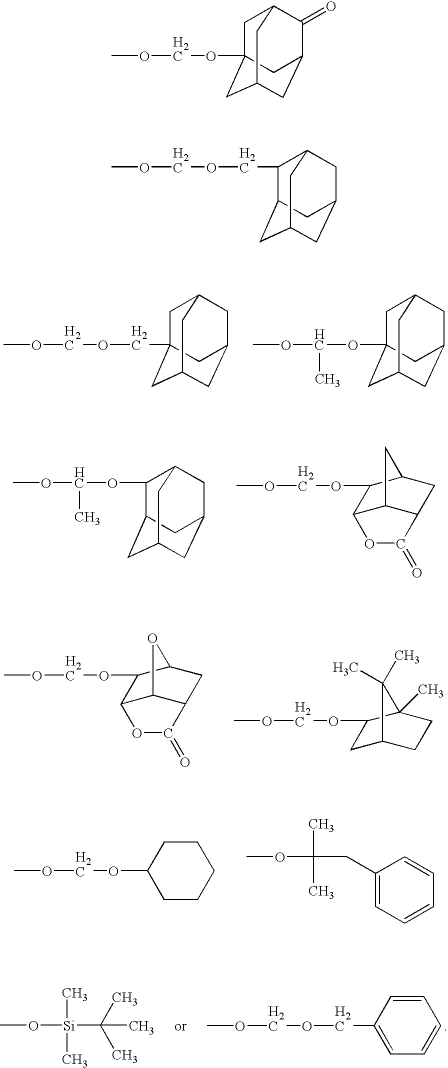

7. The cyclic compound according to claim 1, wherein the dissolution controlling group represented by R¹ and R⁴ is a substituted or unsubstituted linear aliphatic hydrocarbon group comprising 1 to 20 carbon atoms, a substituted or unsubstituted branched aliphatic hydrocarbon group comprising 3 to 12 carbon atoms, a substituted or unsubstituted cyclic aliphatic hydrocarbon group comprising 3 to 20 carbon atoms, a substituted or unsubstituted aromatic group comprising 6 to 10 carbon atoms, an alkoxyalkyl group, a silyl group, or a group formed by bonding at least one substituted or unsubstituted linear aliphatic hydrocarbon group comprising 1 to 20 carbon atoms, substituted or unsubstituted branched aliphatic hydrocarbon group comprising 3 to 12 carbon atoms, substituted or unsubstituted cyclic aliphatic hydrocarbon group comprising 3 to 20 carbon atoms, substituted or unsubstituted aromatic group comprising 6 to 10 carbon atoms, alkoxyalkyl group, or silyl group
with
a divalent group, which is a substituted or unsubstituted alkylene group, a substituted or unsubstituted arylene group, a substituted or unsubstituted silylene group, a group formed by bonding two or more of a substituted or unsubstituted alkylene group, a substituted or unsubstituted arylene group, a substituted or unsubstituted silylene group, or a group formed by bonding at least one substituted or unsubstituted alkylene group, substituted or unsubstituted arylene group, or substituted or unsubstituted silylene group with at least one selected from the group consisting of an ester group, carbonic ester group, and ether group.

8. A photoresist base material comprising the cyclic compound according to claim 1.

9. A photoresist composition, comprising the photoresist base material according to claim 8 and a solvent.

10. The photoresist composition according to claim 9, further comprising a photoacid generator.

11. The photoresist composition according to claim 9, further comprising a basic organic compound as a quencher.

12. An ultrafine processing method comprising adding the photoresist composition according to claim 9 to a semi-conductor material or a semi-conductor precursor material.

13. A semiconductor device prepared by the ultrafine processing method according to claim 12.

14. An apparatus comprising the semiconductor device according to claim 13.

15. The cyclic compound according to claim 2, wherein OR³ is an acid-labile protecting group.

16. The cyclic compound according to claim 15, wherein the acid-labile protecting group is a substituent with a molecular weight of 15 or more and 2000 or less, which has a tertiary aliphatic structure, an aromatic structure, a monocyclic aliphatic structure, or a polycyclic aliphatic structure.

17. An ultrafine processing method, comprising adding a solvent to the photoresist base material according to claim 8.
